SEQUENCE LISTING (41 sequences)

SEQ ID NO: 1 - DNA, 27 bp, Artificial Sequence
  synthetic primer
ggataccgctgtctactactgtagccg

SEQ ID NO: 2 - DNA, 23 bp, Artificial Sequence
  synthetic primer
ctgctcaccgtcaccaatgtgcc

SEQ ID NO: 3 - protein, 10 aa, Artificial Sequence
  synthetic peptide
KLVVVGAGGV

SEQ ID NO: 4 - protein, 10 aa, Artificial Sequence
  synthetic peptide
KLVVVGAVGV

SEQ ID NO: 5 - protein, 10 aa, Artificial Sequence
  synthetic peptide
KLVVVGACGV

SEQ ID NO: 6 - protein, 10 aa, Artificial Sequence
  synthetic peptide
KLVVVGADGV

SEQ ID NO: 7 - protein, 9 aa, Artificial Sequence
  synthetic peptide
VVGAVGVGK

SEQ ID NO: 8 - protein, 9 aa, Artificial Sequence
  synthetic peptide
VVGACGVGK

SEQ ID NO: 9 - protein, 9 aa, Artificial Sequence
  synthetic peptide
KITDFGRAK

SEQ ID NO: 10 - protein, 9 aa, Artificial Sequence
  synthetic peptide
KITDFGLAK

SEQ ID NO: 11 - protein, 9 aa, Artificial Sequence
  synthetic peptide
VVGAGGVGK

SEQ ID NO: 12 - protein, 10 aa, Artificial Sequence
  synthetic peptide
ELAGIGILTV

SEQ ID NO: 13 - protein, 9 aa, Artificial Sequence
  synthetic peptide
LLGRNSFEV

SEQ ID NO: 14 - protein, 245 aa, Artificial Sequence
  synthetic peptide
MASDIQMTQSPSSLSASVGDRVTITCRASQDVNTAVAWYQQKPGKAPKLLIYSASFLYSGVPSRFSGSRSGTDFTLTISSLQPEDFATYYCQQYYSSPPTFGQGTKVEIKRTGGGSGGGGSGGGASEVQLVESGGGLVQPGGSLRLSCAASGFNINSSSIHWVRQAPGKGLEWVASIAPARGSTRYADSVKGRFTISADTSKNTAYLQMNSLRAEDTAVYYCSRNYYAYTAMDVWGQGTLVTVSS

SEQ ID NO: 15 - protein, 234 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQYSSSPPTFGQGTKVEIKRTGGGSGGGASEVQLVESGGGLVQPGGSLRLSCAASGFNINTSYIHWVRQAPGKGLEWVASIYPNDGYNRYADSVKGRFTISADTSKNTAYLQMNSLRAEDTAVYYCSRAAYAMDVWGQGTLVTVSS

SEQ ID NO: 16 - protein, 238 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQSYYSPPTFGQGTKVEIKRTGGGSGGGASEVQLVESGGGLVQPGGSLRLSCAASGFNINNTYIHWVRQAPGKGLEWVASIYPTDGYTRYADSVKGRFTISADTSKNTAYLQMNSLRAEDTAVYYCSRTYYSYYSAMDVWGQGTLVTVSS

SEQ ID NO: 17 - protein, 243 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQYYSYPPTFGQGTKVEIKRTGGGSGGGGSGGGASEVQLVESGGGLVQPGGSLRLSCAASGFNITSSYIHWVRQAPGKGLEWVAYISPEDGYARHADSVKGRFTISADTSKNTAYLQMNSLRAEDTAVYYCSRDDTYYYSAMDVWGQGTLVTVSS

SEQ ID NO: 18 - protein, 243 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGGGASEVQLVESGGGLVQPGGSLRLSCAASGFNISTSYIHWVRQAPGKGLEWVATIDPNDGYSRYADSVKGRFTISADTSKNTAYLQMNSLRAEDTAVYYCSRTNNTAADAMDVWGQGTLVTVSS

SEQ ID NO: 19 - protein, 241 aa, Artificial Sequence
  synthetic peptide
DIQMTQSPSSLSASVGDRVTIACRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGGGASEVQLVESGGGLVQPGGSLRLSCAASGFHINGSYIHWVRQAPGKGLKWVAYIDPETGYSRYADSVKGRFAISADMSKNTAYLQMNSLRAEDTAVYYCSRDSASDAMDVWGQGTLVTVSS

SEQ ID NO: 20 - protein, 241 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGGGASEVQLVESGGGLVQPGGSLRLSCAASGFNINGSYIHWVRQAPGKGLEWVAYIDPETGYSRYADSVKGRFTISADTSKNTAYLQMNSLRAEDTAVYYCSRDSASDAMDVWGQGTLVTVSS

SEQ ID NO: 21 - protein, 240 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGASEVQLVESGGGLVQPGGSLRLSCAASGFNINSNYIHWVRQAPGKGLEWVAYITPETGYYRYADSVKGRFTISADTSKNTAYLQMNSLRAEDTAVYYCSRNYYSAYAMDVWGQGTLVTVSS

SEQ ID NO: 22 - protein, 240 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGASEVQLVESGGGLVQPGGSLRLSCAASGFNINSNYIHWVRQAPGKGLEWVAYITPETGYYHYADSVKGRFTISADTSKNTAYLQMNSLRAEDTAVYYCSRNYYSAYAMDVWGQGTLVTVSS

SEQ ID NO: 23 - protein, 240 aa, Artificial Sequence
  synthetic peptide
DIQMTQSPSSLSASVGDRVTITCRASQDVNTAVAWYQQKPGKAPKLLIYGASFLYSGVPSRFSGSRSGTDFTLTISSLQPEDFATYYCQQYYYYPPTFGQGTKVEIKRTGGGSGGGGSGASEVQLVESGGGLVQPGGSLRLSCAASGFYINSNYIHWVRQAPGKGLEWVAYITPETGYYHYADSVKGRFTISADTSKNTAYLQMNSLRAEDTAVYYCSRNYYSAYAMDVWGQGTLVTVSS

SEQ ID NO: 24 - protein, 240 aa, Artificial Sequence
  synthetic peptide
DIQMSQSPSSLSASVGDRVTITCRTSQDANTAVAWYQQKPGKAPKLLFYSASFLFSGVPSRFSGSRSGTDFTLTISSLQPEDFATYYCQQYYYYPPTFGQGTKVEIKRTGGGSGGGGSGASEVQLVESGGGLVQPGGSLRLSCAASGFNINSNYIHWVRQAPGKGLEWVAYITPETGYYRYADSVKGRFTISADTSKNTAYLQMNSLRAEDTAVYYCSRNYYSAYAMDVWGQGTLVTVSS

SEQ ID NO: 25 - protein, 234 aa, Artificial Sequence
  synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQSYSSPPTFGQGTKVEIKRTGGGSGGGASEVQLVESGGGLVQPGGSLRLSCAASGFNINDTYIHWVRQAPGKGLEWVAYISPASGNSRYADSVKGRFTISADTSKNTAYLQMNSLRAEDTAVYYCSRSYAAMDVWGQGTLVTVSS

SEQ ID NO: 26 - protein, 9 aa, Artificial Sequence
  synthetic peptide
KVYEGVWKK

SEQ ID NO: 27 - protein, 9 aa, Artificial Sequence
  synthetic peptide
TTAPFLSGK

SEQ ID NO: 28 - protein, 9 aa, Artificial Sequence
  synthetic peptide
IMQLMPFGC

SEQ ID NO: 29 - protein, 10 aa, Artificial Sequence
  synthetic peptide
LIMQLMPFGC

SEQ ID NO: 30 - protein, 9 aa, Artificial Sequence
  synthetic peptide
KITDFGRAK

SEQ ID NO: 31 - protein, 10 aa, Artificial Sequence
  synthetic peptide
KLVVVGAVGV

SEQ ID NO: 32 - protein, 10 aa, Artificial Sequence
  synthetic peptide
GMNWRPILTI

SEQ ID NO: 33 - protein, 241 aa, Artificial Sequence; identical to SEQ ID NO: 20
  synthetic antibody

SEQ ID NO: 34 - protein, 241 aa, Artificial Sequence; identical to SEQ ID NO: 19
  synthetic antibody

SEQ ID NO: 35 - protein, 243 aa, Artificial Sequence; identical to SEQ ID NO: 17
  synthetic antibody

SEQ ID NO: 36 - protein, 9 aa, Artificial Sequence
  synthetic peptide
ATAPSLSGK

SEQ ID NO: 37 - protein, 241 aa, Artificial Sequence; identical to SEQ ID NO: 20
  scFv sequences

SEQ ID NO: 38 - protein, 241 aa, Artificial Sequence; identical to SEQ ID NO: 19
  scFv sequences

SEQ ID NO: 39 - protein, 243 aa, Artificial Sequence; identical to SEQ ID NO: 17
  scFv sequences

SEQ ID NO: 40 - protein, 243 aa, Artificial Sequence
  single chain variable regions
DIQMTQSPSSLSASVGDRVTITCRASQDVNTAVAWYQQKPGKAPKLLIYSASFLYSGVPSRFSGSRSGTDFTLTISSLQPEDFATYYCQQSYYSPPTFGQGTKVEIKRTGGGSGGGGSGGGASEVQLVESGGGLVQPGGSLRLSCAASGFNITSSYIHWVRQAPGKGLEWVAYISPADGYNRYADSVKGRFTISADTSKNTAYLQMNSLRAEDTAVYYCSRTDSTAYTAMDVWGQGTLVTVSS

SEQ ID NO: 41 - protein, 243 aa, Artificial Sequence
  single chain variable regions
DIQMTQSPSSLSASVGDRVTITCRASQDVNTAVAWYQQKPGKAPKLLIYSASFLYSGVPSRFSGSRSGTDFTLTISSLQPEDFATYYCQQYYSYPPTFGQGTKVEIKRTGGGSGGGGSGGGASEVQLVESGGGLVQPGGSLRLSCAASGFNINSSYIHWVRQAPGKGLEWVAYISPTDGYYRYADSVKGRFTISADTSKNTAYLQMNSLRAEDTAVYYCSRTSDTSYAAMDVWGQGTLVTVSS

(19) United States
(12) Patent Application Publication
(10) Pub. No.: US 2018/0086832 A1
(43) Pub. Date: Mar. 29, 2018

(54) HLA-RESTRICTED EPITOPES ENCODED BY SOMATICALLY MUTATED GENES

(71) Applicant: The Johns Hopkins University, Baltimore, MD (US)

(72) Inventors: Bert Vogelstein, Baltimore, MD (US); Kenneth W. Kinzler, Baltimore, MD (US); Shibin Zhou, Owings Mills, MD (US); Luis Diaz, Ellicot City, MD (US); Nickolas Papadopoulos, Towson, MD (US); Andrew Skora, Baltimore, MD (US); Jackie Douglass, Memphis, TN (US); Michael S. Hwang, Seattle, WA (US)

(21) Appl. No.: 15/560,241

(22) PCT Filed: Mar. 23, 2016
(86) PCT No.: PCT/US2016/023673

§ 371 Date: Sep. 21, 2017

Related U.S. Application Data
(60) Provisional application No. 62/186,455, filed on Jun. 30, 2015.
(60) Provisional application No. 62/136,843, filed on Mar. 23, 2015.

(51) Int. Cl. C07K 16/28; C07K 16/32; C07K 16/00; G01N 33/574

(52) U.S. Cl. CPC C07K 16/2833; C07K 16/32; C07K 16/005; G01N 33/574; C07K 2317/32; C07K 2317/92; C07K 2317/55; C07K 2317/24; G01N 2333/70539; C07K 2317/734; C07K 2317/622

(57) ABSTRACT

Mutant epitopes encoded by cancer genes are virtually always located in the interior of cells, making them invisible to conventional antibodies. We generated single chain variable fragments (scFvs) specific for mutant peptides presented on the cell surface by human leukocyte antigen (HLA) molecules. These scFvs can be converted to full-length antibodies, termed MANAbodies, targeting “Mutation Associated Neo-Antigens” bound to HLA. A phage display library representing a highly diverse array of single-chain variable fragment sequences was first designed and constructed. A competitive selection protocol was then used to identify clones specific for peptides bound to pre-defined HLA types. In this way, we obtained scFvs, including one specific for a peptide encoded by a common KRAS mutant and another by a common EGFR mutant. Molecules targeting MANA can be developed that specifically react with mutant peptide-HLA complexes even when these peptides differ by only one amino acid from the normal, wild-type form.

This invention was made with government support under CA 43460 and CA 062924 awarded by National Institutes of Health. The government has certain rights in the invention.

### TECHNICAL FIELD OF THE INVENTION

This invention is related to the area of antibody generation. In particular, it relates to constructs that contain an antibody variable region in a single chain or other types of antibody molecules.

### BACKGROUND OF THE INVENTION

Cancers are the result of sequential mutations of oncogenes and tumor suppressor genes (1). In theory, somatic mutations are ideal therapeutic targets because they are not found in virtually any normal cell (2). Even though the protein products of these mutations generally only subtly differ from the wild type (wt) form, often by a single amino acid, this difference is sufficient for effective targeting. When the protein is an enzyme, such as that encoded by BRAF, the resulting structural change can provide a pocket for the binding of specific enzymatic inhibitors (3-5). Antibodies are one of the most successful types of modern pharmaceutical agents and have been shown to be able to specifically recognize proteins that differ only by a single amino acid or by the modification of a single amino acid (5-11). However, all antibodies used in the clinic are directed against cell surface or secreted proteins rather than intracellular proteins. Intracellular proteins are not accessible to large molecules such as antibodies, but unfortunately, the vast majority of the abnormal epitopes encoded by mutant genes are not on the cell surface (2).

Intracellular antigens, such as viral components, can be recognized by the immune system, though this recognition is based on recognition of proteolytically-processed peptides complexed to human leukocyte antigen (HLA) molecules on the cell surface (12). Indeed, 10% to 20% of the epitopes created by mutant genes in cancers (hereinafter referred to as MANAs, for Mutation-Associated Neo-Antigens) are predicted to bind to common HLA types (12). Moreover, examples of T-cells that can bind to such peptide-HLA complexes have been found in patients as well as in experimental animals (13-16).

The majority of T cell responses generated in vivo against MANAs are “private,” i.e., directed against mutant epitopes encoded by passenger mutations that are present in cancers of individual patients or mice but are not commonly found in patients and do not drive neoplastic growth (2). Immunologic agents targeting such antigens are only useful for the treatment of the individual patients harboring the particular MANA (16-20).

There is a continuing need in the art to identify new therapeutic, diagnostic, and analytic agents for diseases including but not limited to cancer.

### SUMMARY OF THE INVENTION

According to one aspect of the invention an isolated molecule comprising an antibody variable region is provided. The antibody variable region specifically binds to a complex of a human leukocyte antigen (HLA) molecule, a β-2-microglobulin molecule, and a peptide which is a portion of a protein. The peptide comprises a mutant residue which is in an intracellular epitope of the protein. The molecule does not specifically bind to the HLA molecule when the HLA molecule is not in the complex. The molecule also does not specifically bind to the peptide in its wild-type form. Optionally, the molecule does not specifically bind to the peptide when not presented within an HLA complex. The isolated molecules can be used for detecting or monitoring cancer cells or for treating cancers.

According to another aspect of the invention a method is provided for selecting from a nucleic acid library an scFv or Fab or TCR that specifically binds to a complex of (a) a human leukocyte antigen (HLA) molecule, (b) a β-2-microglobulin molecule, and (c) and a first form of a peptide portion of a protein. The first form comprises a mutant residue, and the mutant residue is in an intracellular epitope of the protein. The scFv or Fab or TCR does not specifically bind to the HLA molecule when the HLA molecule is not in the complex. The scFv or Fab or TCR does not specifically bind to the peptide in its wild-type form. The method comprises a step of: positively selecting for scFv or Fab or TCR that bind to said complex in the presence of a competitor complex that comprises (a) a second form of the peptide portion bound to (b) HLA and (c) β-2-microglobulin. The second form is selected from the group consisting of a wild-type form and a peptide with a different mutant residue from the first form. During optional successive performance of the step, amounts of said complex and the competitor complex may be varied so that ratio of competitor complex to relevant complex increases.

According to yet another aspect of the invention a method is provided for selecting from a nucleic acid library an scFv or Fab or T cell receptor that specifically binds to a first form of a peptide portion of a protein. The first form comprises a mutant residue, that is in an intracellular epitope of the protein. The scFv or Fab or TCR does not specifically bind to the peptide in its wild-type form. The method comprises a step of: positively selecting for scFv or Fab or T cell receptors that bind to the first form in the presence of a competitor second form of the peptide portion, wherein the second form is selected from the group consisting of a wild-type form and a peptide with a different mutant residue than the first form.

These and other embodiments which will be apparent to those of skill in the art upon reading the specification provide the art with agents for accessing epitopes which are ordinarily intracellular but which are displayed on the surface of particular cells in disease conditions.

### BRIEF DESCRIPTION OF THE DRAWINGS

FIG. 1. Generation of MANAbody. The process of MANAbody generation is outlined with the competitive phage selection highlighted at the center.

FIG. 2A-2E. Selective binding of phage and purified scFv to mutant monomers. Monomers folded with the indicated peptides, beta-2-microglobulin, and HLA molecules were incubated with phage clones or purified scFv at different dilutions, followed by ELISA with anti-M13 (for phage) or anti-Flag tag (for scFv) antibody. (FIG. 2A) Selective binding of phage clones collected and expanded after the final selection phase for KRAS(G12V)-HLA-A2 binders. Clone D10 is highlighted by the red arrow. (FIG. 2B) Selective binding of phage clone D10 to different monomers. ****, P<0.0001, comparing KRAS(G12V)-HLA-A2 against every other monomer at 1:80 dilution. (FIG. 2C) Selective binding of the purified D10 scFv to different monomers. ****, P<0.0001 comparing KRAS(G12V) HLA-A2 against every other monomer at 1 μg/mL dilution. (FIG. 2D) Selective binding of phage clone C9 to different monomers. ****, P<0.0001, comparing EGFR(L858R)-HLA-A3 against every other monomer at 1:900 dilution. (FIG. 2E) Selective binding of the purified C9 scFv to different monomers. ****, P<0.0001, comparing EGFR(L858R)-HLA-A3 against every other monomer at 1 μg/mL dilution. (B-E), monomers folded with wt or specified mutant peptides and HLA molecules are shown on the x-axis. ELA: negative control peptide, No Monomer: well coated with streptavidin without monomer attached.

FIG. 3A-3D. Selective binding of candidate phage clones or purified D10 scFv to cells displaying mutant peptides on the cell surface. T2 or T2A3 cells were pulsed with indicated peptides and then incubated with D10 phage (FIG. 3A), purified D10 scFv (FIG. 3B), or C9 phage (FIG. 3C) before analysis of the stained cells by flow cytometry. ELA, LLG: negative control peptides; for C9 phage, KRAS(WT) was used as a negative control peptide. (FIG. 3D), scFv-mediated, complement-dependent cell killing. CDC assay was performed by incubating T2 cells with 10% rabbit complement and D10 scFv or D10-7 scFv preconjugated to anti-V5 antibody, after T2 cells were pulsed or unpulsed or pulsed with the indicated peptides. CellTiter-Glo® was used to assess the viability of cells. ***P<0.001, comparing KRAS(G12V)/D10-7 to all other points at the 0.66 nM (−0.18 on x-axis) antibody concentration; ns, not significant (P=0.488), comparing KRAS(WT)/D10-7 to Unpulsed/D10-7 at the 0.66 nM antibody concentration.

FIG. 4A-4B. Selective affinity of D10 MANAbody. (FIG. 4A) Selective binding of D10 MANAbody to KRAS(G12V)-HLA-A2. Monomers folded with indicated peptides and HLA molecules were incubated with D10 MANAbody at different dilutions, followed by ELISA with anti-human IgG antibody. ***, P<0.0001 comparing KRAS G12V HLA-A2 against every other monomer at 1 μg/mL dilution. (FIG. 4B) Selective binding of D10 MANAbody to cells displaying mutant peptides on the cell surface. T2 cells were unpulsed or pulsed with indicated peptides and then incubated with D10 MANAbody or with an isotype control antibody, before analysis of the stained cells by flow cytometry.

FIG. 5. Linear presentation of scFv/M13 pIII open reading frame in the phagemid. pelB, pelB periplasmic secretion signal; V_{L} and V_{H}, light and heavy chains in scFv; myc, myc tag; TEV, TEV protease cleavage recognition sequence; M13 pIIIl, M13 pIII coat protein.

FIG. 6A-6C. Flowchart of competitive selection. The selection process consisted of 10 rounds of selection and amplification, which were divided into three phases: enrichment phase (FIG. 6A; rounds 1-3), competitive phase (FIG. 6B; rounds 4-8), and final selection phase (FIG. 6C; rounds 9-10). Ratio of mutant (MUT) monomer to wild type (WT) competitive monomer used in each competitive round is shown.

FIG. 7A-7B. Binding of phage after different selection phases. Monomers folded with the indicated peptides and HLA molecules were incubated with phage (en masse) at different dilutions, followed by ELISA with anti-M13 antibody. (FIG. 7A) Binding of phage collected after the enrichment phase. (FIG. 7B) Binding of phage collected after the final selection phase. KRAS(G12V), KRAS peptides with G12V mutations; KRAS(WT), wild type KRAS peptide.

FIG. 8. Purified D10 scFv does not bind KRAS peptides not complexed with HLA molecules or denatured monomers. Biotinylated KRAS peptides alone, native monomers, or heat denatured monomers were incubated with purified scFv at different dilutions, followed by ELISA with anti-Flag tag antibody. KRAS(G12V), KRAS peptides with G12V mutation; KRAS(WT), wild type KRAS peptide; No Monomer, well coated with streptavidin without monomer attached.

FIG. 9. Selective binding of purified D10-7 scFv to different monomers. Monomers folded with the indicated peptides, beta-2 microglobulin, and HLA molecules were incubated with D10-7 scFv at different dilutions, followed by ELISA with anti-Flag tag antibody. The peptide is shown on the line below the bar graph and the HLA protein type bound to the monomer is shown on the line below the peptide. ****, P<0.0001 comparing KRAS(G12V)-HLA-A2 against every other monomer at 0.037 μg/mL dilution.

FIG. 10. Flowchart of modified competitive selection yielding the C9 phage. The selection process consisted of 9 rounds of selection and amplification, which were divided into three phases: enrichment phase (rounds 1-5), competitive phase (rounds 6-8), and final selection phase (round 9). Ratio of mutant (MUT) monomer to wild type (WT) competitive monomer used in each competitive round is shown.

FIG. 11A-E. Peptide loading efficiency as assessed by W6/32 antibody staining. T2 or T2A3 cells were unpulsed or pulsed with indicated peptides and then incubated with the W6/32 antibody, before analysis of the stained cells by flow cytometry. ELA: control peptide. FIG. 11A: KRAS(G12V), FIG. 11B: KRAS(WT), FIG. 11C: EGFR(L858R), FIG. 11D: EGFR(WT), FIG. 11E: ELA control

FIG. 12. Selective binding of D10 phage to cells displaying mutant peptides on the cell surface. T2 cells were pulsed with indicated peptides and then incubated with D10 phage, C9 phage as control, or no phage before analysis of the stained cells by flow cytometry. KRAS(G12V), KRAS peptides with G12V mutation; KRAS(WT), wild type KRAS peptide; ELA, irrelevant peptide.

FIG. 13. Selective binding of C9 phage to cells displaying mutant peptides on the cell surface. T2A3 cells were pulsed with indicated peptides and then incubated with C9 phage or no phage before analysis of the stained cells by flow cytometry. EGFR(L858R), EGFR peptide with L858R mutation; EGFR(WT), wild type EGFR peptide; KRAS(WT), wild type KRAS peptide.

FIG. 14. W6/32 antibody-mediated, complement-dependent cell killing. CDC assay was performed by incubating T2 cells with the W6/32 antibody and 10% rabbit complement, after T2 cells were pulsed or unpulsed with indicated peptides. CellTiter-Glo® was used to assess the viability of cells.

FIG. 15. Selective binding of D10 MANAbody to cells displaying mutant peptides on the cell surface. T2 cells were pulsed with indicated peptides and then incubated with or without D10 MANAbody before analysis of the stained cells by flow cytometry. A control antibody (7A) was also used.

FIG. 16 shows an Enzyme Linked Immunosorbant Assay using beta catenin S45F specific scFvs presented on bacteriophage. CTNNB1 S45F scFv candidate (E10): Phage ELISA (normalized). Legend indicates dilutions of phage used. Monomers and phage clone used labeled on the X-axis. * indicates identical sequences. ELISA using E10 or G7 expressing phage. Both scFvs show significantly more binding to the Mutant epitope (CTNNB1 S45F) than the WT. ELISA using E10 or G7 expressing phage. Both scFvs show significantly more binding to the Mutant epitope (CTNNB1 S45F) than the WT. Both scFvs show increased binding to the mutant epitope HLA-A3 complex when compared to the wild-type complex. See Example 11.

FIG. 17 shows results of a flow cytometric assay of E10 CTNNB S45F phage staining. Phage clone is specific to CTNNB S45F peptide-pulsed cells. scFv is directed against CTNNB1 (Beta Catenin) S45F mutation. W6/32 data shows an increase in antibody binding over b2m (negative control) showing that the mutant and wild-type peptide can be presented on HLA-A3 complexes present on the T2A3 cell line. E10 phage staining shows that the scFv binds specifically to S45F epitopes (80,400 k MFUs) over control peptides (600-800 MFU). Rows labeled 1-5 demonstrate that both the mutant and wild-type beta catenin epitopes can be presented on cell surface HLA-A3 complexes. Rows labeled 6-9 show the mean fluorescent intensity (MFI) of the E10 phage bound to T2A3 cells pulsed with the indicated peptide. E10 phage recognizes the mutant peptide specifically, and does not bind to cell surface presented complexes pulsed with either the wild-type or control (K3WT) peptide. The histogram provides an alternative representation of the specificity of the E10 phage. See Example 11.

FIG. 18 shows results of a complement dependent cytotoxicity assay (CDC). CDC with CTG (Cell Titer Glo™) on T2A3 Cells with E10 (CTNNB1 S45F HLA-A3) scFv:anti-V5 Conjugate. The decrease in relative luciferase units seen in the E10:V5 conjugate series under with S45F pulsed cell condition demonstrates specific killing of cells presenting the beta catenin S45F mutation. This assay is conducted as described in Proc Natl Acad Sci USA. 2015 Aug. 11; 112(32):9967-72, with the exception that only a single antibody concentration is used (10 ug/ml). T2A3 cells were left unpulsed with peptide or pulsed with either wild-type or mutant beta catenin peptide. When incubated with complement sera only, the cells pulsed with peptide underwent CDC dependent cell death. See Example 11.

FIG. 19. Combined EGFR T790M 9-mer and 10-mer Hits (14 unique): Phage Supernatant and Precipitated Phage ELISA. Two experiments (phage supernatant and precipitated phage ELISA) shown left and right, respectively. Legend indicates monomers (HLA type) tested; the data demonstrate high specificity of the phage for both the 9 and 10 amino acid epitopes predicted to present this mutation. The figure shows results of an ELISA testing either phage supernatant or precipitated phage as indicated. Certain phage clones including D3E6, D2D8 and D2D6 were further analyzed after supernatant testing. High specificity was observed for all three candidates. The EGFR T790 control peptide is not a biologically relevant (wild-type) control, rather it is a sequence highly similar to the T790M 9-mer that was used for competitive panning. See Examples 12 and 13.

FIGS. 20 (table) and 21 (histogram) show results of samples labeled 1-7 in the table and demonstrate that both the mutant EGFR T790M epitopes (9-mer and 10-mer) can be presented on cell surface HLA-A2 complexes. Rows labeled 8-10 in the table of FIG. 20 show the mean fluorescent intensity (MFI) of the D3E6 phage bound to T2 cells pulsed with the indicated peptide. D3E6 phage recognizes the mutant peptide specifically, and does not bind to cell surface presented complexes pulsed with control peptides. The histogram (FIG. 21) is an alternative representation showing the specificity of the D3E6 phage. The D3E6 phage stains mutant but not wild type 790 peptide-pulsed cells, and does not stain ELA (negative HLA-A2 control peptides). See Examples 12 and 13.

FIGS. 22-25 (table in FIG. 22 and flow cytometry in FIGS. 23-25) provide further confirmation that the D3E6, D2D8, and D2D6 clones recognize the mutant peptides specifically, and do not bind to cell surface presented complexes pulsed with control peptides. FIG. 23: EGFR T790M clone 1 (“D3E6”) staining of T2 cells; FIG. 24: EGFR T790M clone 2 (“D2D8”) staining of T2 cells; FIG. 25: EGFR T790M clone 3 (“D2D6”) staining of T2 cells. See Examples 12 and 13.

FIG. 26 shows results of an ELISA which tested precipitated phage. High specificity for KRAS G12V in HLA-A2 was observed for the F10 candidate. Legend indicates dilutions of phage used. Monomers used are indicated on the X-axis. See Example 10.

FIG. 27 provides a histogram of results from a flow cytometry assay showing that F10 affinity matured variants can specifically recognize the mutant KRAS epitope pulsed on T2 cells over the wild-type control. See Example 10.

### DETAILED DESCRIPTION OF THE INVENTION

We have developed an approach to generate and identify antibody variable regions that selectively target complexes containing common HLA types bound to peptide products of commonly mutated oncogenes. As these HLA-peptide complexes are expected to be exclusively present on the surface of cancer cells or other disease related cells, antibodies targeting them could in principle be used for therapeutic or monitoring purposes. These same approaches for identifying antibody variable regions can also be used to identify T cell receptors.

Mutations in oncogenes and tumor suppressor genes drive tumorigenesis, and their protein products form therapeutic targets that are absent from normal cells. However, nearly all such mutant epitopes lie in the interior of the cells, either in the cytoplasm or nucleus, complicating immunotherapies directed against the mutants. The antibody variable regions and T cell receptors described here overcome the shielding of intracellular targets lying within cells by targeting forms that are displayed on the surface of cells. Nonetheless, the methods and approaches described here may be used not solely for tumor suppressors and oncogenes, but also passenger mutations (not drivers of carcinogenesis) as well as for other proteins that are the product of somatic mutagenesis or which are expressed on cell surfaces as the result of somatic mutagenesis.

Examples of intracellular proteins which may be targeted include without limitations EGFR, KRAS, NRAS, HRAS, p53, PIK3CA, ABL1, beta-catenin, and IDH1/2. In order to have widest applicability, it is desirable to select mutations that are prevalent in cancer populations. Examples of such mutations include those in residues EGFR L858, KRAS G12, KRAS G13, HRAS G12, NRAS G12, HRAS Q61, NRAS Q61, IDH1 R132, beta-catenin S45, IDH2 R140, and IDH2 R172. Common mutants include EGFR L858R, KRAS G12V, KRAS G12C, KRAS G12D, HRAS Q61P, NRAS Q61P, HRAS Q61R, NRAS Q61R, HRAS Q61K, NRAS Q61K, EGFR T790M, IDH1 R132H, beta-catenin S45F, IDH2 R140Q, and IDH2 R172K. However, even a private or personal disease specific mutation coding for an epitope that is intracellular may be the target of an scFv or Fab or T cell receptors.

Libraries which can be made and screened include any that produce useful specific binding molecules, such as scFv, Fab, and TCR. The complexity of the repertoire of binding molecules is preferably very high. The libraries may be made in any suitable vector system, including but not limited to M13 phage, ribosomes, and yeast. For Fab libraries see Lee et al., J Mol Biol. “High-affinity human antibodies from phage-displayed synthetic Fab libraries with a single framework scaffold,” 2004 Jul. 23; 340:1073-93. For T cell receptor libraries see Kieke et al., “Selection of functional T cell receptor mutants from a yeast surface-display library,” Proc Natl Acad Sci USA. 1999; 96: 5651-5656. For ribosome display libraries see Stafford et al., Protein Eng Des Sel. “In vitro Fab display: a cell-free system for IgG discovery.” 2014; 27:97-109. Libraries may be made using synthetic oligonucleotides, synthetic trimers, or synthetic deoxyribonucleotides, for example. Each option permits biasing of the mixtures to bias the ultimate library composition.

The rarity of the desired scFv or Fab or TCR in a library is in some part due to the nature of the desired target. The desired target comprises a complex of an HLA molecule, a β-2-microblobulin protein, and a peptide. However, of this whole complex, the desired scFv or Fab or TCR will only recognize a particular epitope that contains a mutant residue, most likely a substitution of one amino acid for another. Moreover, it will not specifically recognize the same macromolecular complex in which the residue is wild-type. Because of this extremely narrow focus, a strong selection process is required, in addition to an extraordinary amount of diversity in the library. A positive selection step for the desired scFv or Fab or T cell receptors has been devised which is performed in the presence of a competitor complex. The competitor complex comprises wild-type form of the peptide bound to HLA and β-2 microglobulin. Alternatively, the competitor complex may comprise a peptide with a highly similar sequence to the mutant peptide, such as a peptide with one or more additional mutant residues or a peptide with an alternate, non-wild-type, residue at the same residue as the mutant peptide. The positive selection agent comprises HLA, β-2-microglobulin, and the “mutant” peptide. Optionally, the competitive selection step will be performed repeatedly. As the step is repeatedly performed, the ratio of competitor complex to positive selection agent can be increased. Optionally, the competitive panning is followed by a negative selection step using the competitor complex. Optionally the competitive complex and/or the positive selection agent may be displayed or expressed on the surface of a cell for the selection step. In an alternative aspect of the invention, this type of selection process may be used to pan for binding molecules that recognize a single amino acid difference in a protein or peptide that is not part of an HLA/β-2 microglobulin complex. In a further option, the peptide does not represent an intracellular epitope.

The HLA molecule which is used to present peptide with a mutant residue may be from any HLA gene (A, B, C, E. F, and G) and allele of those genes. More prevalent genes and alleles, such as HLA-A2, HLA-A3, and HLA-B7, will find wider usage among human patients of some groups. Other HLA genes which may be used are HLA DP, DM, DOA, DOB, DQ, and DR.

When useful molecules are identified that specifically bind to a complex of (1) an HLA molecule, (2) a β-2-microglobulin, and (3) a peptide comprising a mutant residue (found in an intracellular epitope in the full native protein) they can be used for various purposes and in various derivatives. The molecules can be bound or attached to a detectable label. Detectable labels can be any that are known in the art including, without limitation, radionuclides, chromophores, enzymes, and fluorescent molecules. Such molecules can be used, for example, to monitor anti-tumor therapy or to detect cancer cells in a sample, or to diagnose cancer. The molecules can alternatively be bound, conjugated, or attached to a therapeutic agent. Such therapeutic agents can be specifically targeted to cells expressing the protein by means of the scFv or Fab or T cell receptor identified. Another derivative of the identified molecule that may be usefully made is a chimeric antigen receptor (CAR). This derivative includes as part of a single protein, the identified molecule comprising an antibody variable region, a hinge region, a transmembrane region, and an intracellular domain. See, e.g., Curran et al., “Chimeric antigen receptors for T cell immunotherapy: current understanding and future directions,” J. Gene Med 2012; 14: 405-415. The CDR sequences of a useful molecule may be incorporated into an intact antibody, to form a MANAbody, as described in the examples. Alternatively, the useful molecule is not a part of an intact antibody molecule. The useful molecule may also be included as part of a chimeric protein with another scFv/antibody, such as an anti-CD3 scFv, to form a bispecific targeting agent. Such a chimeric protein may be used to target T cells to the tumor, inducing anti-tumor activity.

Any diagnostic technique known in the art, particularly any immunological diagnostic technique, can be used with the useful molecules. They can be used on samples that are tissue samples or tissue homogenates, for example. They can be used in immunohistochemistry, ELISA, immunoprecipitation, immunoblots, etc. Detection will be dependent on the detectable label that is attached or used to identify immune complexes. Any detection technique can be used. Therapeutic administration can be accomplished using any known means suitable for administering an antibody or specific binding molecule. Administration may be by injection or infusion into the peripheral circulatory system, for example, or intratumoral, intraspinal, intracerebellar, intraperitoneal, etc.

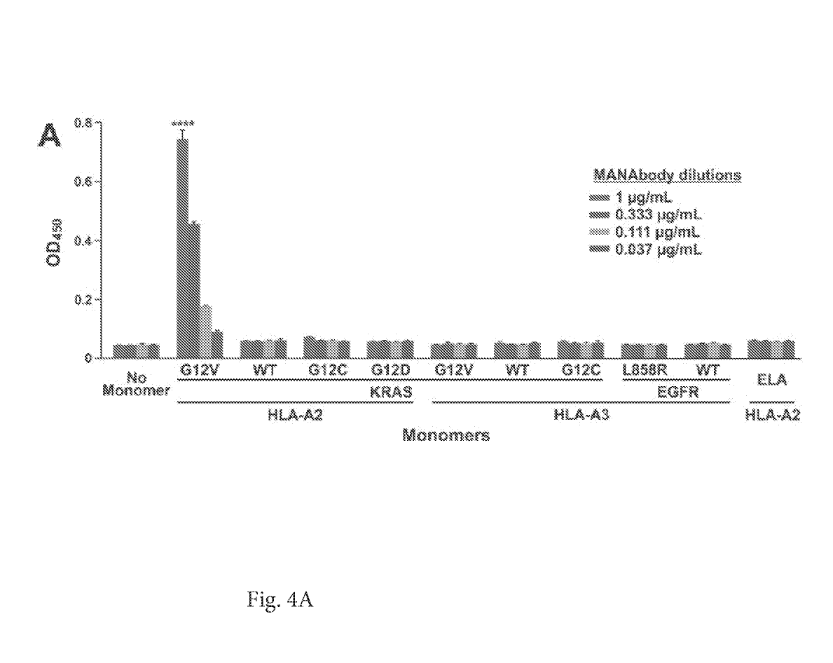

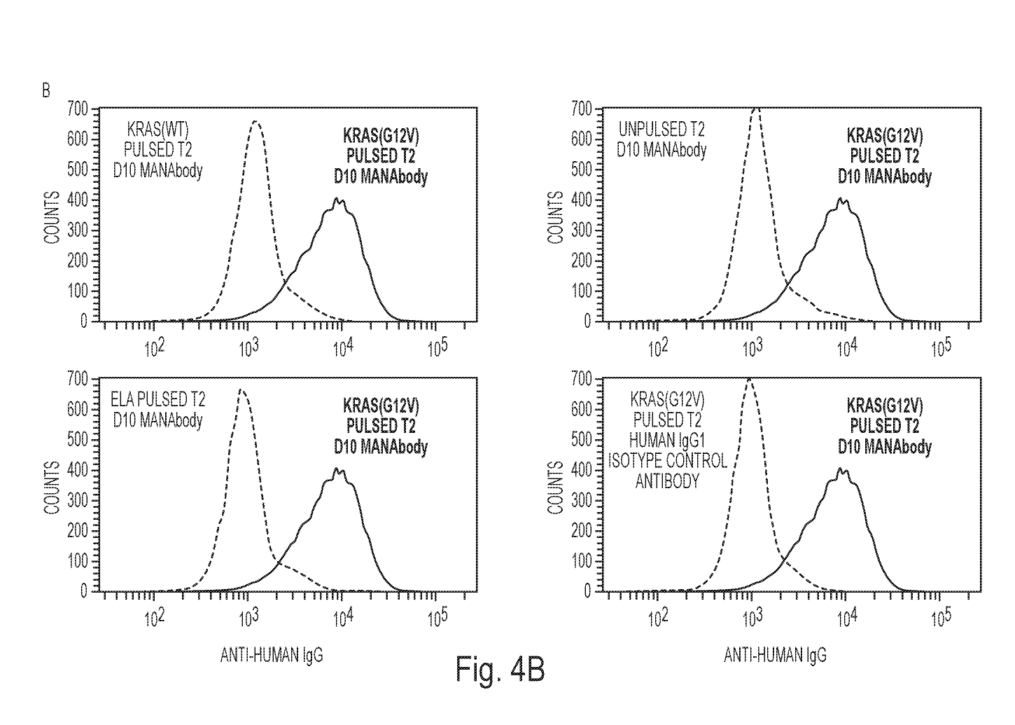

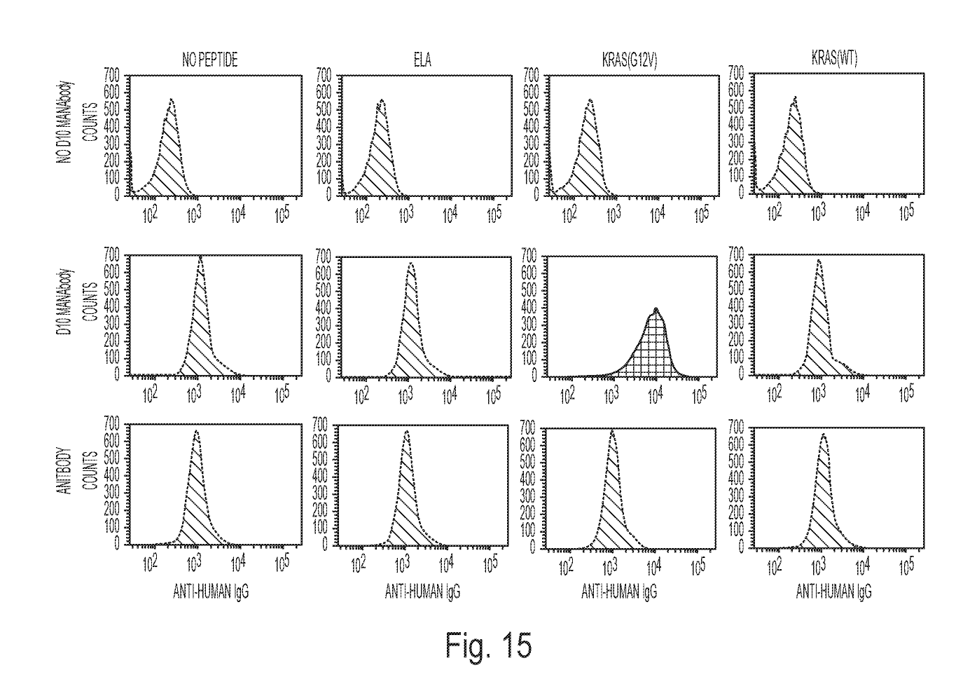

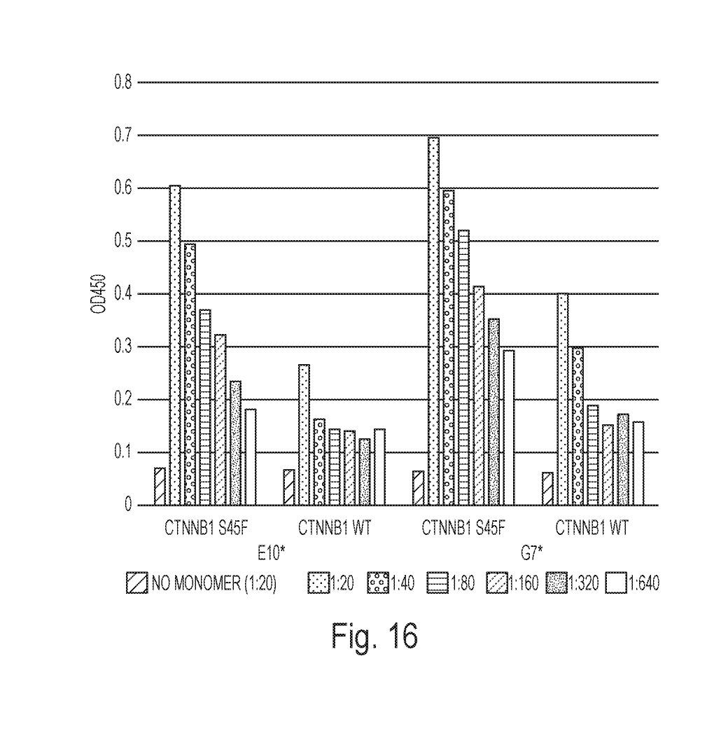

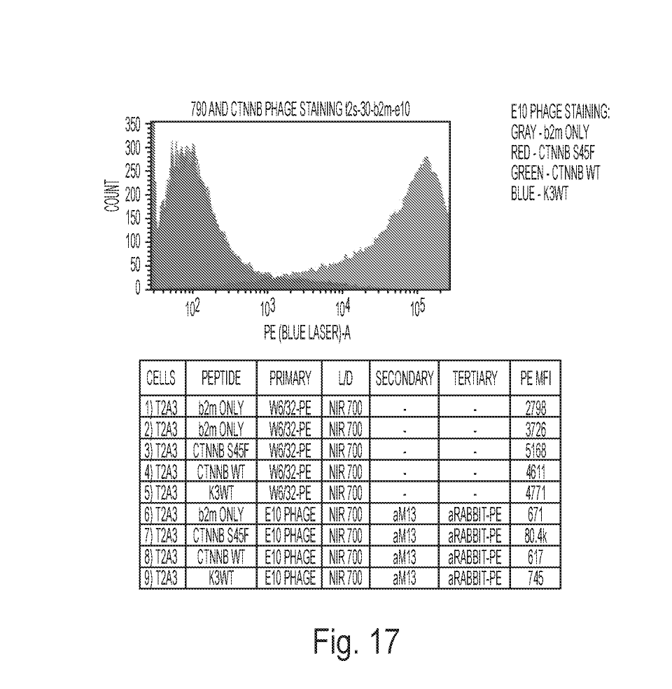

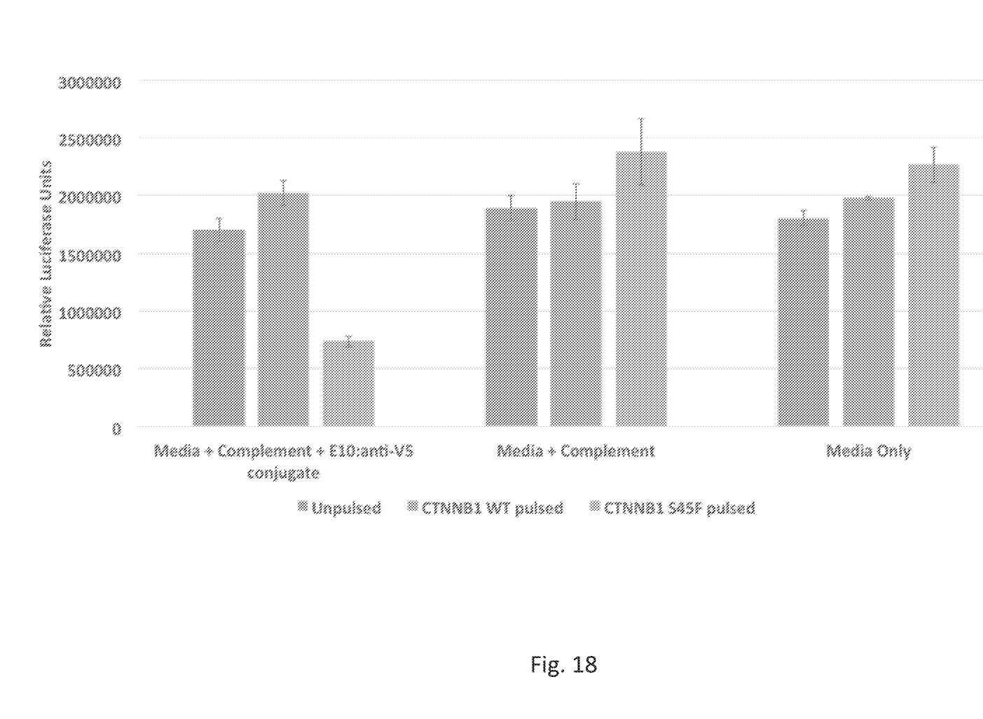

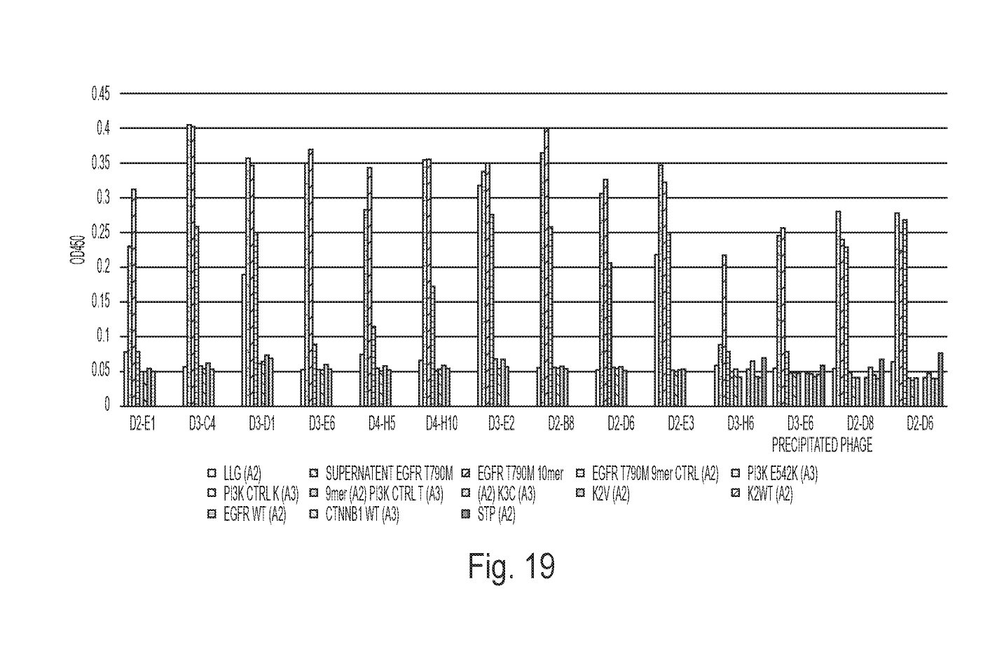

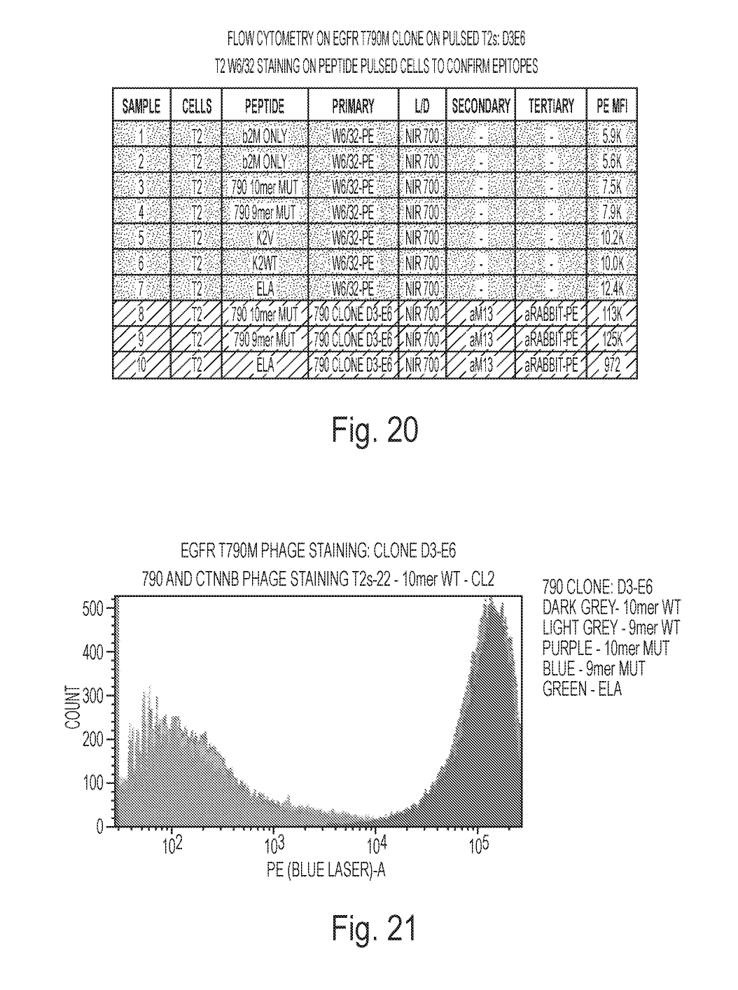

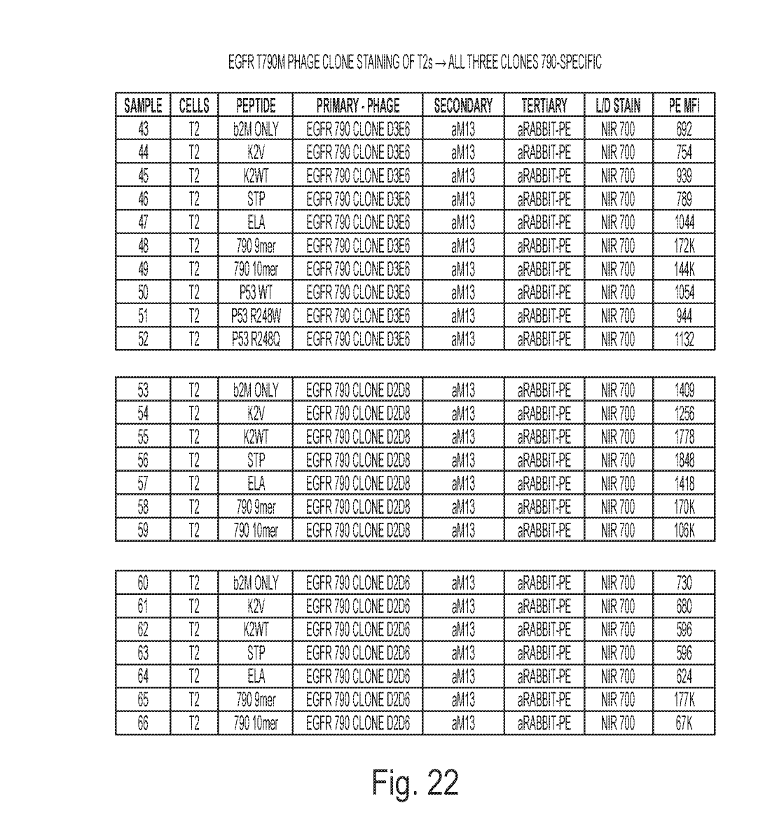

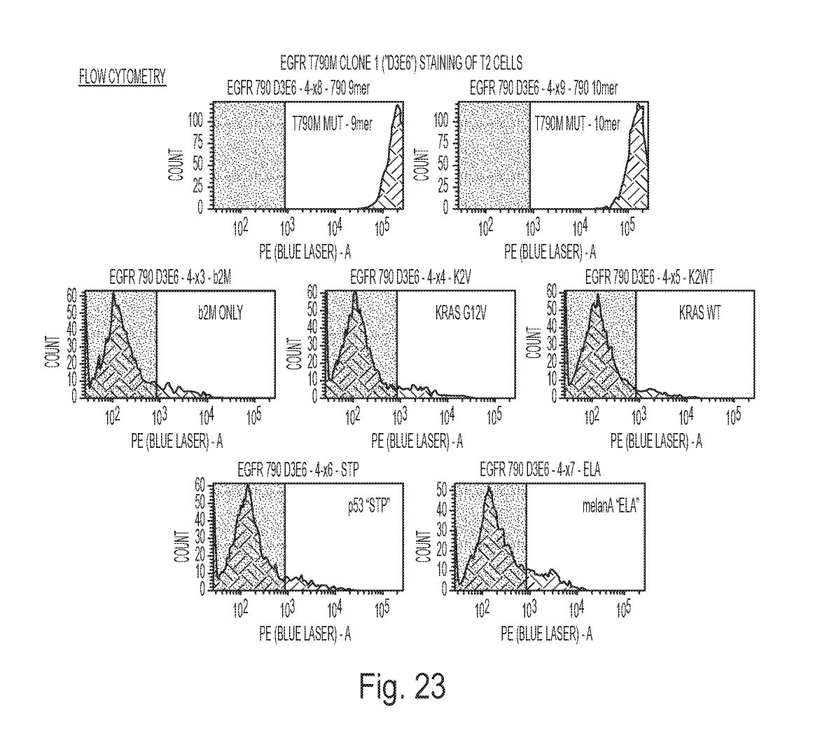

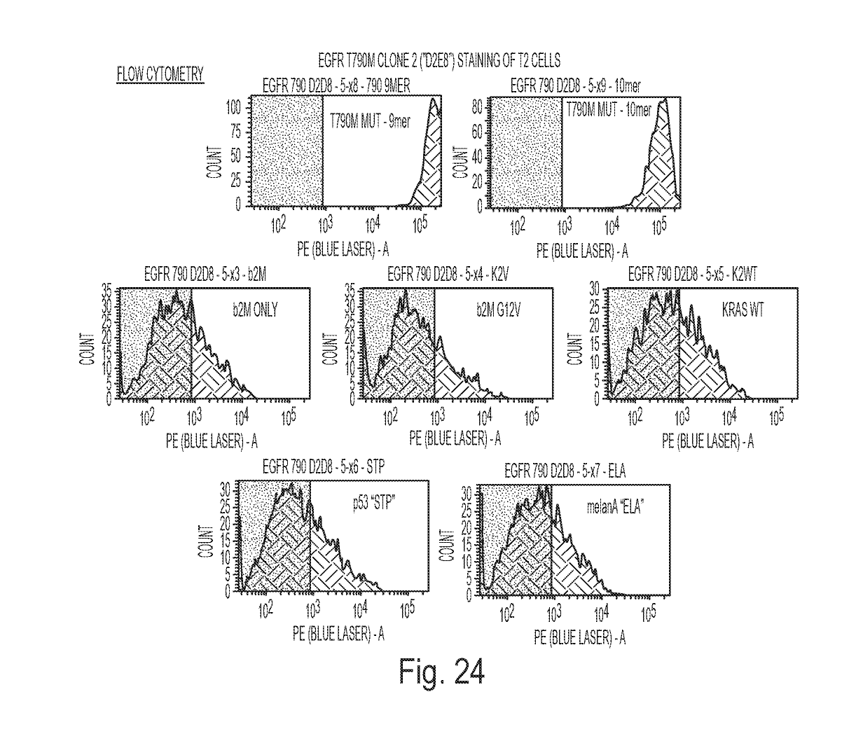

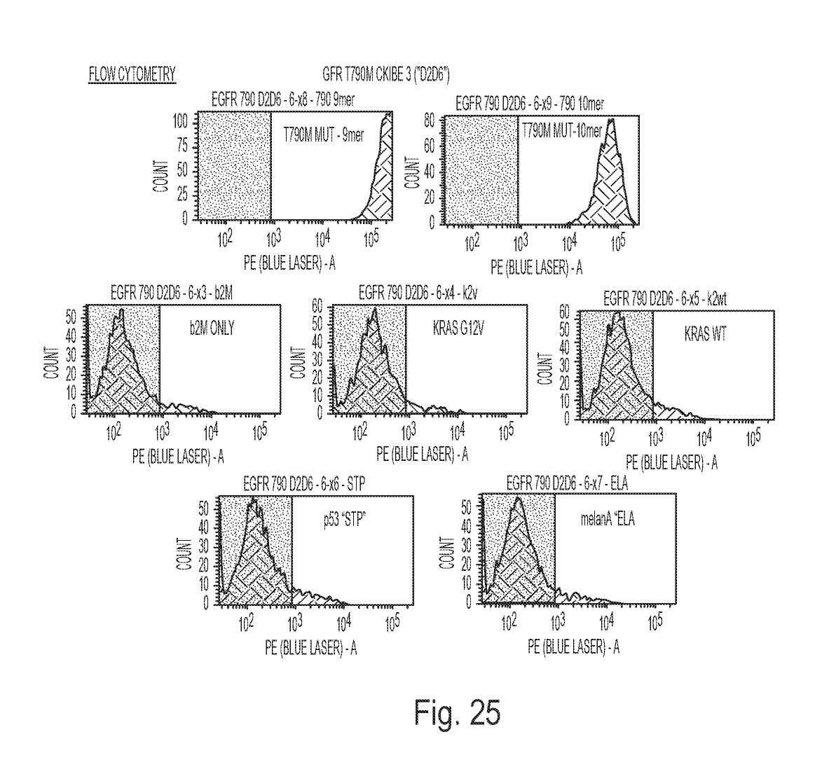

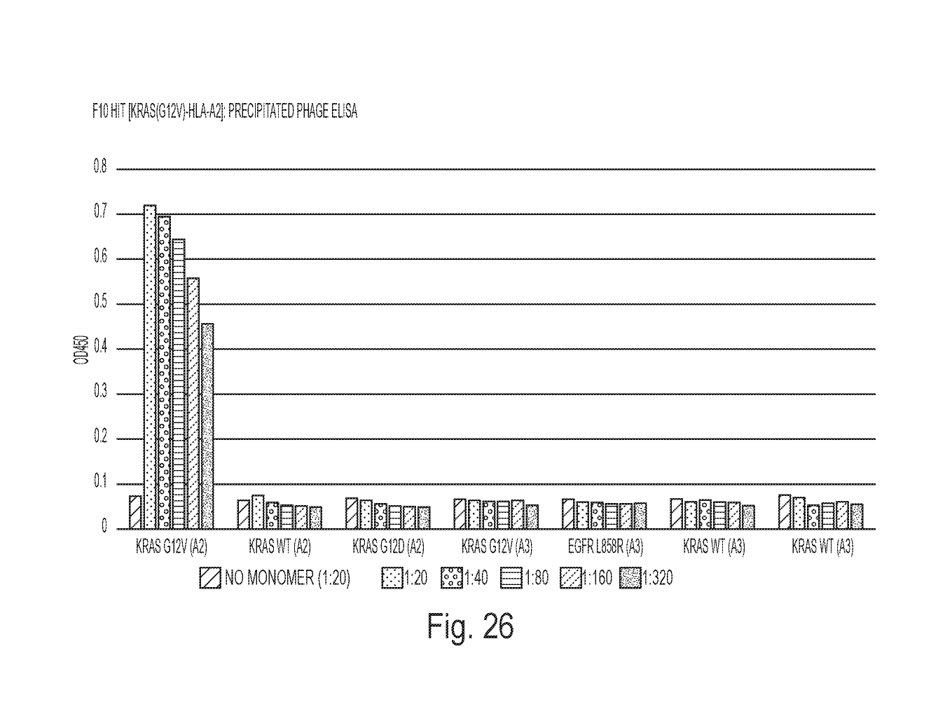

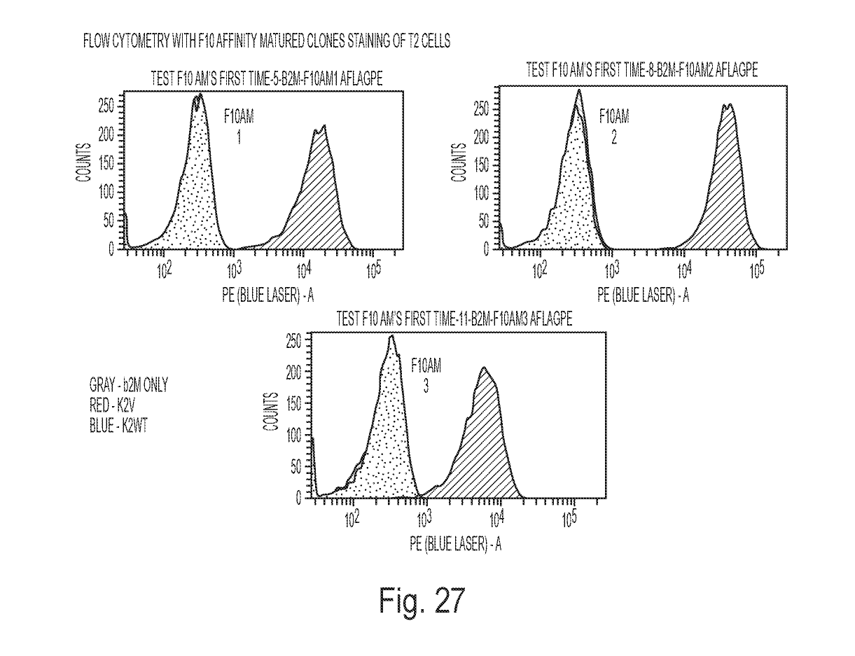

We have established a procedure for generating scFvs that selectively bind to mutant peptides embedded within HLA-beta-2 microglobulin complexes. Using this procedure, we obtained scFvs against the products of two commonly mutated oncogenes (KRAS and EGFR) when complexed with two common HLA types (A2 and A3, respectively). These scFvs bind to the peptide-HLA complexes on the surface of cells and can kill those cells when complement is present. Converting an scFv into a complete, bivalent antibody containing the Fc region sometimes results in loss of affinity (46, 47). However, we successfully generated a complete antibody using the D10 scFv sequence, and this MANAbody retained the specificity of the scFv (FIG. 4B, FIG. 15). We have not yet attempted to generate a MANAbody using the C9 scFv directed to the mutant EGFR peptide complexed with HLA-A3.

Other antibodies, termed TCRmimics, have been generated against peptide-HLA complexes in the past (48-49). A first important aspect of our study is the generation of antibody-based reagents that differentially recognize HLA complexes containing peptides varying only by a single amino acid. A second important aspect of our study is that the variant peptides are commonly found in human cancers.

The greatest challenge in both cancer diagnosis and therapy is specificity—developing reagents that recognize or kill cancer cells but not normal cells. The relative lack of specificity currently presents a major obstacle for the wider implementation of powerful immunotherapeutic agents such as chimeric antigen receptors and bi-specific antibodies (57-60). In this context, specific somatic mutations that alter the encoded proteins of cancer driver genes represent biochemical features that distinguish cancer cells from normal cells in unparalleled fashion. The strength of the work described here is that it demonstrates the feasibility of generating highly specific reagents that recognize these altered proteins in a context that is clinically relevant (cell surface). This sets the stage for further exploration of such reagents and their incorporation into suitable diagnostic and therapeutic vehicles.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Materials and Methods

Cell Lines. T2 cells (ATCC, Manassas, Va.) were cultured in RPMI-1640 (ATCC) with 10% FBS (GE Hyclone, Logan, Utah, USA), 1% penicillin streptomycin (Life Technologies), and 20 IU/mL recombinant human IL-2 (Proleukin™, Prometheus Laboratories) at 37° C. under 5% CO₂. T2A3 cells (a kind gift from the Eric Lutz and Liz Jaffee, JHU) were grown in the same conditions as T2 cells but also with the addition of 500 ug/mL Geneticin (Life Technologies) and 1× Non-Essential Amino Acids (Life Technologies).

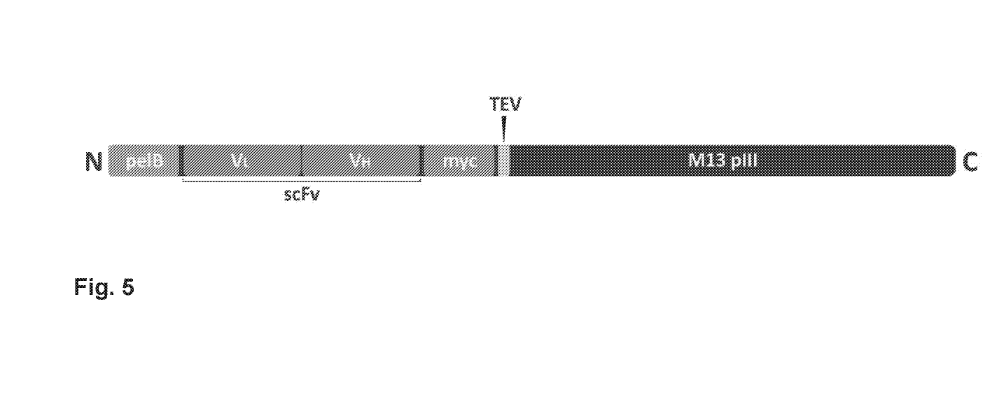

Phage Display Library Construction. Oligonucleotides were synthesized at DNA 2.0 (Menlo Park, Calif.) using mixed and split pool degenerate oligonucleotide syntheses. The oligonucleotides were incorporated into the pADL-10b phagemid (Antibody Design Labs, San Diego, Calif.). This phagemid contains an F1 origin, and a transcriptional repressor unit consisting of a lac operator and a lac repressor to limit uninduced expression. The scFv was synthesized with a pelB periplasmic secretion signal and was subcloned downstream of the lac operator. A myc epitope tag followed by a TEV protease cleavage recognition sequence was placed immediately downstream of the variable heavy chain, which was followed in frame by the full length M13 pIII coat protein sequence. (FIG. 5) Successful cloning was confirmed by Sanger sequencing 45 random clones obtained from transformation of a small portion of the ligated product. Twenty-four of the clones contained the expected sequences or silent mutations, 4 contained in-frame mutations within the framework regions, and 17 contained deletions of one or more base pairs, indicating a successful synthesis and cloning fraction of 53%. This was later confirmed following library electroporation as discussed below.

Ten ng of the ligation product was mixed on ice with 10 μL of electrocompetent SS320 cells (Lucigen, Middleton, Wis.) and 14 μL of double-distilled water (ddH2O). This mixture was electroporated using a Gene Pulser electroporation system (Bio-Rad, Hercules, Calif.) and allowed to recover in Recovery Media (Lucigen) for 60 min at 37° C. Cells transformed with 60 ng of ligation product were pooled and plated on a 24-cm×24-cm plate containing 2×YT medium supplemented with carbenicillin (100 μg/mL) and 2% glucose. Cells were grown at 37° C. for 6 hours and placed at 4° C. overnight. To determine the transformation efficiency for each series of electroporations, aliquots were taken and titered by serial dilution. Cells grown on plates were scraped into 850 mL of 2×YT medium with carbenicillin (100 μg/mL) plus 2% glucose for a final OD₆₀₀ of 5-15. Two mL of the 850 mL culture were taken and diluted ˜1:200 to reach a final OD₆₀₀ of 0.05-0.07. To the remaining culture, 150 mL of sterile glycerol was added before snap freezing to produce glycerol stocks. The diluted bacteria were grown to an OD₆₀₀ of 0.2-0.4, infected with M13K07 Helper phage (NEB, Ipswich, Mass. or Antibody Design Labs) at an MOI of 1 and allowed to recover at 37° C. for 30 min before shaking at 37° C. for an additional 30 min. The culture was centrifuged and the cells were resuspended in 2×YT medium with carbenicllin (100 μg/mL) and kanamycin (50 μg/mL) and grown overnight at 30° C. for phage production. The following morning, the bacterial culture was aliquoted into 50 mL Falcon tubes and pelleted twice, first at 3000 g and then at 12000 g, to obtain clarified supernatant. The phage-laden supernatant was precipitated on ice for 40 min with a 20% PEG-8000/2.5 M NaCl solution at a 1:4 ratio of PEG/NaCl:supernatent. After precipitation, phage from each 50 mL-culture was centrifuged at 12,000 g for 40 minutes and resuspended in a 1 mL vol 1× TBS, 2 mM EDTA. Phage from multiple tubes were pooled, re-precipitated, and resuspended to an average titer of 1×10¹³ cfu/mL in 15% glycerol. The total number of transformants obtained was determined to be 5.5×10⁹. The library was aliquoted and stored in 15% glycerol at −80° C.

Next-generation sequencing of the complete phage library. DNA from the library was amplified using the following primers (Forward: GGATACCGCTGTCTACTACTGTAGCCG, SEQ ID NO: 1 Reverse: CTGCTCACCGTCACCAATGTGCC, SEQ ID NO: 2) which flank the CDR-H3 region. Additional molecular barcode sequences were incorporated at the 5′-ends of these primers to facilitate unambiguous enumeration of distinct phage sequences. The protocols for PCR-amplification and sequencing are previously published in (1). Sequences were processed and translated using a custom SQL database and both the nucleotide sequences and amino acid translations were analyzed using Microsoft Excel.

Peptides and HLA-Monomers. A wt KRAS peptide (KLVVVGAGGV; SEQ ID NO: 3) predicted to bind to HLA-A2, a mutant KRAS (G12V) peptide (KLVVVGAVGV; SEQ ID NO: 4) predicted to bind to HLA-A2, a mutant KRAS (G12C) peptide (KLVVVGACGV; SEQ ID NO: 5) predicted to bind to HLA-A2, a mutant KRAS (G12D) peptide (KLVVVGADGV; SEQ ID NO: 6) predicted to bind to HLA-A2, a mutant KRAS (G12V) peptide (VVGAVGVGK; SEQ ID NO: 7) predicted to bind to HLA-A3, a mutant KRAS (G12C) peptide (VVGACGVGK; SEQ ID NO: 8) predicted to bind to HLA-A3, a mutant EGFR (L858R) peptide (KITDFGRAK; SEQ ID NO: 9) predicted to bind to HLA-A3, a wt EGFR peptide (KITDFGLAK; SEQ ID NO: 10) predicted to bind to HLA-A3, and a wt KRAS peptide (VVGAGGVGK; SEQ ID NO: 11) predicted to bind to HLA-A3, and negative HLA-A2 control peptides ELA (ELAGIGILTV; SEQ ID NO: 12) and LLG (LLGRNSFEV; SEQ ID NO: 13) were synthesized at a purity of >90% by Peptide 2.0 (Chantilly, Va.). Peptides were resuspended in DMSO at 10 mg/mL and stored at −80° C. HLA-A2 and HLA-A3 monomers were synthesized by refolding recombinant HLA with peptide and beta-2 microglobulin, purified by gel-filtration, and biotinylated (Fred Hutchinson Immune Monitoring Lab, Seattle, Wash.). Monomers were confirmed to be folded prior to selection by performing an ELISA using W6/32 antibody (BioLegend, San Diego, Calif.), which recognizes only folded HLA(59). A rabbit anti-HLA-A antibody EP1395Y (Abcam, Cambridge, Mass.), which recognizes both folded and unfolded HLA, was used as a control for binding of unfolded monomers to the ELISA plates.

Selection for phages binding to mutant KRAS-HLA-A2. Biotinylated monomers containing HLA and beta-2-microglobulin proteins were conjugated to either MyOne T1 streptavidin magnetic beads (Life Technologies, Carlsbad, Calif.) or to streptavidin agarose (Novagen, Millipore, Darmstadt, Germany). The biotinylated monomers were incubated with either 25 μL of MyOne T1 beads or 100 μL of streptavidin agarose in blocking buffer (PBS, 0.5% BSA, 0.1% Na-azide) for 1 hr at room temperature (RT). After the initial incubation, the complexes were washed 3 times with 1 ml blocking buffer and resuspended in 100 uL blocking buffer.

Enrichment phase: The enrichment phase of selection consists of rounds 1 to 3. In round one, 1.4×10¹² phage (140 uL), representing 250-fold coverage of the library, were incubated for 30 minutes in a mixture of 25 ul washed naked MyOne T1 beads and 1 ug (100 uL) heat-denatured HLA-A2 conjugated to MyOne T1 beads. It should be noted that after heat-denaturation, only the biotinylated HLA molecule, but not the peptide or beta-2-microglobulin, will be able to bind the MyOne T1 beads. This step is referred to as “negative selection,” necessary to remove any phage recognizing either streptavidin or denatured monomer, present to a small extent in every preparation of biotinylated monomer. After this negative selection, beads were immobilized with a DynaMag-2 magnet (Life Technologies) and the supernatant containing unbound phage was transferred for positive selection against the mutant KRAS-HLA-A2 monomer. The amount of monomer was decreased from 1 ug in round 1 to 500 ng in round 2 and 250 ng in round 3 and phage were incubated for 30 minutes. Prior to elution, beads were washed 10 times with 1 ml, 1× TBS containing 0.05%, 0.1%, and 0.25% Tween-20 in rounds 1 to 3 respectively. Phage were eluted by resuspending the beads in 1 mL of 0.2 M glycine, pH 2.2. After a 10-minute incubation, the solution was neutralized by the addition of 150 μL of 1 M Tris, pH 9.0. Eluted phage were used to infect 10 mL cultures of mid-log-phase SS320s, with the addition of M13K07 helper phage (MOI of 4) and 2% glucose. Bacteria were then incubated as previously described and the phage were precipitated the next morning with PEG/NaCl.

Competitive phase: In the competitive phase (rounds 4 to 8), negative selection incorporated the same heat-denatured HLA-A2 monomer and naked streptavidin-coated magnetic beads, but also incorporated 1 ug of native HLA-A3 monomer. After negative selection, beads were isolated with a magnet and the supernatant containing unbound phage was transferred for competitive selection. This was performed by co-incubating phage with mutant KRAS-HLA-A2 monomer conjugated to magnetic streptavidin-coated magnetic MyOne T1 beads in the presence of wt KRAS-HLA-A2 monomer conjugated to streptavidin-coated agarose beads (Novagen EMD Millipore, Darmstadt, Germany). The ratio of mutant monomer to wt monomer was dropped 2-fold each round, from 1:1-1:32, holding the amount of the wt monomer constant at 1 μg. Prior to elution, beads were washed 10 times in 1 ml 1× TBS containing 0.5% Tween-20. Phage were eluted and used to infect mid-log phase SS320 cells as described above for the enrichment phase.

Final selection phase: In the final selection phase (rounds 9 -10), 1 μg each of denatured and native KRAS-(WT)-HLA-A2 monomers was used for negative selection to remove residual wt monomer-binding phage. After negative selection, beads were immobilized with a magnet and the supernatant containing unbound phage was transferred for positive selection with 62.5 ng of mutant KRAS-HLA-A2 monomer, as described for the enrichment phase above.

Selection for phage binding to mutant EGFR-HLA-A3. This was performed as described above for the isolation of phages binding to mutant KRAS-HLA-A2, but with the following modifications. Selection was initiated with 2.5×10¹² input phage and the number of input phage was decreased over the course of selection (see below). To increase expression of the scFv-pIII fusion protein in the early rounds, IPTG was employed to de-repress the lac operon. IPTG was added at an initial concentration of 10 μM through round three, and subsequently lowered to 5 μM for the remaining 6 rounds of selection. Additionally, in rounds 1 to 8, negative selection was performed with 2 μg of heat-denatured biotinylated HLA-A2 and HLA-A3 conjugated to streptavidin magnetic beads (MyOne T1) as well as 25 uL naked streptavidin-coated magnetic beads. In the competitive phase, the ratio of mutant to wt monomers was gradually decreased from 1:2 to 1:64 and the amount of phage added was gradually reduced from 2.5×10¹² to 10×10⁶.

Affinity Maturation. Affinity maturation of D10 was performed at AxioMx as follows. Briefly, the D10 scFv sequence (SEQ ID NO: 37) was synthesized and used as template for error-prone PCR-based mutagenesis. The resulting mutagenized library underwent three rounds of selection and amplification where the phage was negatively selected against KRAS(WT)-HLA-A2 monomer prior to positive selection against KRAS(G12V)-HLA-A2 monomer and subsequent amplification. Following selection and amplication, potential phage were isolated, sequenced, and tested via ELISA. To identify higher affinity D10 variants. Eight clones were identified as having higher affinity to KRAS(G12V)-HLA-A2 with no KRAS(WT)-HLA-A2 binding, of which one clone D10-7 (SEQ ID NO: 38) was chosen for further characterization.

ELISA. Streptavidin-coated, 96-well plates (Thermo Scientific, Walthan, Mass.) were coated with a 200 nM solution of biotinylated monomers in blocking buffer (PBS with 0.5% BSA, 2 mM EDTA, and 0.1% sodium azide) at 4° C. overnight. Plates were briefly washed with 1× TBST (TBS+0.05% Tween-20). Phage were serially diluted to the specified dilutions in 1× TBST and 100 uL was added to each well. Phage were incubated for 1 hr at RT, followed by vigorous washing (6 washes with 1× TBST using a spray bottle (Fisher Scientific, Waltham, Mass.). The bound phage were incubated with 100 μL of rabbit anti-M13 antibody (Pierce, Rockford, Ill.) diluted 1:2000 in 1× TBST for 1 hr at RT, followed by washing an additional 6× times and incubation with 100 μL of anti-Rabbit IgG-HRP (Jackson Labs, Bar Harbor, Me.) diluted 1:10,000 in 1× TBST for 45 min at RT. After a final 6 washes with 1× TBST, 100 μL of TMB substrate (Biolegend, San Diego, Calif.) was added to the well and the reaction was quenched with 1 N HCl or 2 N sulfuric acid. Absorbance at 450 nm was measured with a SpectraMax Plus 384 plate reader (Molecular Devices, Sunnyvale, Calif.) or a Synergy H1 Multi-Mode Reader (BioTek, Winooski, Vt.).

Monoclonal phage ELISA was performed by selecting individual colonies of SS320 cells transduced with a limiting dilution of phage obtained from the final selection phase. Individual colonies were inoculated into 200 μl of 2×YT medium containing 100 μg/mL carbenicillin and 2% glucose and grown for three hours at 37° C. The cells were then infected with 1.6×10⁷ M13K07 helper phage (Antibody Design Labs, San Diego, Calif.) at MOI of 4 in a dep-well 96-well plate and incubated for 30 min at 37° C. with no shaking followed by 30 min of shaking. The cells were pelleted, resuspended in 300 μL of 2×YT medium containing carbenicillin (100 μg/mL) and kanamycin (50 μg/mL) and grown overnight at 30° C. Cells were pelleted and the phage-laden supernatant was used for ELISA as described above. ELISA with purified scFvs was performed essentially as above, with serial dilutions from a starting concentration of 1 μg/mL and the use of a 1:2000 diluted anti-Flag-HRP antibody (Abcam) for detection. ELISAs with the full-length D10 MANAbody were performed similarly, with serial dilutions from a starting concentration of 1 μg/mL and a 1:2000 diluted anti-human IgG-HRP antibody as the secondary antibody (Life Technologies). Monomer heat denaturation was first performed by diluting monomer into 100 μL ddH2O followed by a 5 minute heat block incubation at 100° C.

scFv Production. Primers were designed to amplify the entire scFv coding region. A Gateway™ directional cloning sequence was added to the forward primer to facilitate subcloning into Gateway™ entry vectors and an AviTag™ sequence was added to the reverse primer to allow for future biotinylation of the recombinant scFv. The clones were sequence verified and recombined into a pET-DEST42 destination vector containing C-terminal V5 and His epitope tags (Life Technologies).

BL21 DE3 Gold cells transformed with recombinant plasmids were, grown in 1 liter batches to an OD₆₀₀ of 1.0 chilled to approximately 20° C. and induced with 500 uM IPTG. Protein was expressed overnight at 20° C. The next morning bacteria were pelleted, resuspended in periplasmic extraction buffer (50 mM Tris pH 7.4, 20% sucrose, 1 mM EDTA, 5 mM MgCl2) and incubated on ice for 30 minutes in the presence of 1/10 volume 1 mg/ml lysozyme. After cells were pelleted at 12,000 g for 30 minutes, the supernatant was filtered through a 22 uM filter (Millipore) and incubated with 1 ml Ni-NTA resin (Qiagen) for one hour. The supernatant and bead mixture were loaded onto a gravity column, washed, and eluted with increasing amounts of imidazole. Samples from each aliquot were run on an SDS polyacrylamide gel and the fractions containing pure protein were dialyzed overnight at 4° C. in 1× PBS pH 7.4. ELISAs were performed per standard protocol to ensure scFv binding capability and specificity using an anti-V5 HRP antibody (Life Technologies).

Alternatively, scFv sequences were provided to AxioMx Inc., subcloned into a vector containing a periplasmic localization sequence, and N-terminal Flag and C-terminal His tags. scFv was then purified via nickel chromatography.

Antibody Production. The scFv sequence was grafted on to the trastuzamab (4D5) sequence for recombinant antibody expression. Both heavy and light chain sequences were provided to Geneart. for codon optimization, synthesis, subcloning, and protein production (Geneart, Life Technologies, Carlsbad, Calif.). An IgG signal sequence was included on each chain for protein expression and antibody secretion using the Expi293™ cell culture system. Following 72 hours of protein expression, antibodies from the one liter culture were purified with column chromatography and eluted in 17 mL PBS aliquoted and shipped at 8.25 mg/mL.

T2 and T2A3 Cell Staining. For peptide pulsing, T2 and T2A3 cells were washed once in 50 mL PBS and once in 50 mL RPMI-1640 without serum before incubation at 5×10⁵ cells per mL in serum-free RPMI-1640 containing 50 μg/mL peptide and 10 μg/mL human beta-2 microglobulin (ProSpec, East Brunswick, N.J.) for 4 hr or overnight at 37° C. The pulsed cells were pelleted, washed once in stain buffer (PBS containing 0.5% BSA, 2 mM EDTA, and 0.1% sodium azide) and resuspended in stain buffer. Phage staining was performed at 4° C. with ˜1×10⁹ phage for 30 min in 200 ul total volume, followed by 3×4 mL rinses in stain buffer by centrifugation at 500 g for 5 min at 6° C. Cells were resuspended in 200 uL stain buffer and stained with 1 uL of rabbit anti-M13 antibody (Pierce, Rockford, Ill.) on ice for 30 min, followed by three rinses with 4 mL stain buffer. Cells were then resuspended in 200 uL stain buffer and incubated with 1 uL anti-rabbit-Alexa Fluor 488™ (Life Technologies) on ice for 30 min, and rinsed an additional three times before analysis. ScFv staining was performed with 1 μg of scFv for 30 min on ice in 100 uL stain buffer, followed by three rinses in stain buffer at 4° C. Cells were then stained with 1 μL of mouse anti-V5-FITC (Life Technologies, Grand Island, N.Y.) for 30 min on ice, followed by three rinses in stain buffer at 6 C. Antibody staining was performed by resuspending cells in 100 uL stain buffer, and blocking with 1 ug goat anti-human antibody (Life Technologies) on ice 30 min, followed by three rinses at 4° C. Cells were resuspended in 200 uL stain buffer, and stained with 5 ug of D10 antibody (or isotype control) for 30 min on ice, followed by three rinses. Cells were resuspended in 200 uL stain buffer, and stained with 2 uL goat anti-human-PE antibody (Life Technologies) for 30 min on ice, followed by three rinses. Peptide pulsing was assessed by incubation of pulsed T2 or T2A3 cells with 5 uL of W6/32-PE (Bilegend) in 100 uL of stain buffer, followed by three washes. Stained T2 and T2A3 cells were analyzed using a FACSCalibur or LSRII flow cytometer (Becton Dickinson, Mansfield, Mass.).

T2 Cell Complement-Dependent Cytotoxicity. scFvs were conjugated to an anti-V5 mouse monoclonal antibody (Life Technologies, Grand Island, N.Y.) at a 2:1 molar ratio overnight at 4° C. Conjuaged scFvs or control anti-HLA antibody W6/32 (Bio-X-Cell) were serially diluted in serum-free RPMI-1640 on ice. Baby rabbit complement (Cedarlane), resuspended with ice cold ddH₂O, was added to the serially diluted antibody conjugates before transferring 60 μL to a 96-well plate. An additional 40 μL of pre-chilled peptide-pulsed T2 cells containing 20,000 cells was transferred to the plate and gently mixed. In all cases, a final complement concentration of 10% was used for the assay. The plate was incubated at 37° C. for 1 hr and subsequently read by the CellTiter-Glo® Luminescent Cell Viability Assay (Promega,) as per manufacturer's instructions. Cell death was calculated by first normalizing each of the three cell subtypes (cells pulsed with different peptides or beta-2 microglobulin-only control) to the maximum luciferase signal (no antibody control) followed by subtraction from 100%. Specific cell death was defined as the cell death at a particular antibody concentration divided by the maximum cell death observed after treatment with W6/32 antibody for each of the given three cell subtypes.

Cell Death (%)=100−(100×Luciferase Signal/Luciferase Signal_{Max})

$$Specific Call Death ( \% ) = \frac{100 - ( 100 Luciferase Signal / Luciferase {Signal}_{Max} )}{100 - ( 100 No Ab Control Signal / W 6 / 32 {Signal}_{Max} )}$$

Affinity and k_{off} Measurements. AlphaScreen® affinity measurements were performed at AxioMx Inc. as follows. Biotinylated KRAS(G12V)-HLA-A2 monomer, unbiotinylated KRAS(G12V)-HLA-A2 monomer, and Flag-tagged D10 scFv were simultanesouly added to a solution containing streptavidin-coated Donor beads with an excitation wavelength of 680 nm and Acceptor beads conjugated with an anti-Flag antibody. Stimulation of Acceptor beads is dependent on proximity of Donor beads and results in an excitation between 520-620 nm when both beads are in close proximity. The EC₅₀, and therefore affinity, of D10 scFv to KRAS(G12V)-HLA-A2 was determined by varying the amounts of competing unbiotinylated monomer and measuring the resulting absorbance.

To conserve limited amounts of antigen and to expedite screening of new scFvs, we developed an off-rate ELISA-based kinetic assay to measure the k_{off}, and therefore half-life, of scFv/monomer disassociation. 100 μL of a 20 nM solution of the respective biotinylated monomer was conjugated to streptavidin-coated 96-well strip plates (R&D Systems). After washing, 100 μL of a 37.0 nM, 9.3 nM, or 2.3 nM solution of D10 scFv, D10-7 scFv, or C9 scFv were added to the plate and allowed to equilibrate on the monomer-coated wells for 2 hr at 22° C. At various timepoints, one strip of the plate was removed, vigorously washed, and placed into 2 liters of 1× TBST with shaking at 22° C. until all timepoints were complete. After the 0 hr timepoint, allowing for a total of 16 hr of scFv dissociation from the time the first strip was washed, all strips were reassembled on the plate and an ELISA was performed as previously described, using an anti-Flag-HRP antibody and TMB substrate. The half-life was determined by fitting an exponential function (Aₜ=Aₒe^{-kt}) to the resulting datapoints following background subtraction, and using the first-order reaction equation t^{1/2}=ln(2)/k. Off-rate estimates for the D10 MANAbody were performed as above with the following exceptions: 100 μL of a 10 nM of monomer was coated on the plate to allow for a 1:2 ratio of streptavidin complexes to monomer, a lower concentration (1.8 nM) of antibody was used, and the assay was carried out to a 32-hr time point.

Statistics. All statistical analyses were performed with Prism 5 (GraphPad Software). Unless otherwise indicated, error bars represent the standard deviation of three technical replicates. Statistical significance was performed with an unpaired, two-tailed t-test.

### EXAMPLE 2

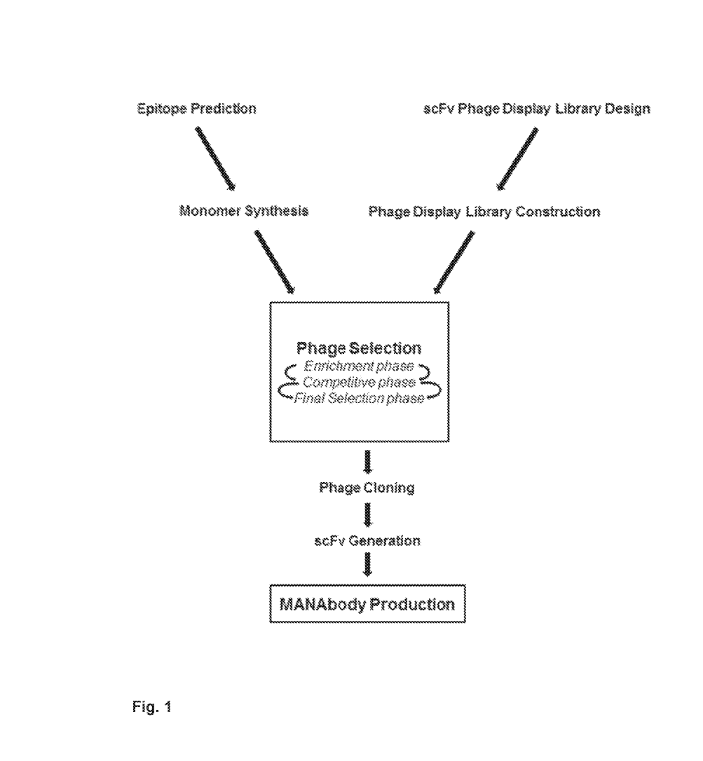

Design and construction of an scFv-based phage display-based scFv library. We began these studies with attempts to generate an antibody against a mutant KRAS peptide in mice (basis for this choice described below). Using conventional approaches to derive monoclonal antibodies after mouse immunization, these efforts failed, as no antibodies specifically reactive with the MANAs were identified. We therefore turned to phage display approaches for generating MANAbodies (FIG. 1). The design of the phage display library followed principles employed in published studies (22) and included some special features. The framework of the library was based on the scFv sequence of humanized 4D5 antibody (Trastuzumab), generated against the protein encoded by ERBB2 (23). This framework was chosen by virtue of its stability on phage and its ease of conversion to a soluble scFv, Fab, or antibody (22, 24). High-resolution crystal structures of the humanized 4D5 have identified the residues within the highly-variable, complementarity-determining regions (CDRs) that play the most significant role in antigen binding (25). This allowed us to focus variability on the most important residues for antigen binding rather than backbone residues. In our library, amino acid substitutions were limited to defined paratope residues in four CDRs, CDR-L3, CDR-H1, CDR-H2 and CDR-H3 (26). We skewed the library to include specific amino acids within the CDRs that either were previously demonstrated to play a significant role in antigen binding or were enriched in naturally occurring antibodies. One important randomization was at CDR-L3, which contained a mixture of serines and tyrosines, two amino acids previously shown to facilitate a minimalist approach to library design (26, 27). CDRs in the heavy chain have been shown to play a more significant role in antigen-binding diversity (28-30), and we therefore introduced more degeneracy in the heavy chain CDRs than in the light chain CDRs. Additionally, at locations wherein increased diversity was sought, the more commonly used IUB nucleotides (designated NNK and NNS degenerate codons) were substituted by DMT codons; this eliminated unwanted residues. In all cases, we employed degenerate nucleotide mixtures intended to minimize the incorporation of sequences resulting in cysteine or stop codons. Finally, we introduced length polymorphisms in CDR-H3, allowing for a stem-loop binding length diversity of 7 amino acids. These changes resulted in a calculated theoretical library complexity of 5×10¹³.

The synthesized oligonucleotide library was cloned into a phagemid vector for scFv expression. This scFv carried a myc tag and was fused to the bacteriophage M13 pill coat protein through a tobacco etch virus (TEV) cleavage site (FIG. 5). This design facilitated purification of scFvs from the phage particles and provided an alternative elution method, accomplished via TEV cleavage, during the subsequent phage-selection process. After library synthesis, 45 clones were sequenced by the Sanger method. The sequencing showed a library success rate of 53%, as defined by the absence of mutations within the framework region and the presence of the expected amino acids within the CDRs. Library diversity was calculated based on transformation efficiencies achieved during library construction, resulting in an estimated diversity of 5.5×10⁹. To further assess the quality of the library, the library was subjected to massively parallel sequencing (31). This analysis revealed 3,785,138 unique clones (46.5% of all clones analyzed). The sequenced region included only the CDR-H3 and not the other three CDRs (CDR-L3, CDR-H1, and CDR-H2) that were systematically varied. The fraction (46.5%) of unique clones therefore represents a minimum estimate of the diversity. Translation of a random subset of sequences further showed the expected amino acid distribution as well as length diversity in CDR-H3.

### EXAMPLE 3

Target selection and competitive strategy for identifying selectively reactive phage clones. We chose MANAbody targets based on the frequency of particular mutations and the strength of their predicted binding to HLA alleles. KRAS is one of the most commonly mutated genes in human cancers, with mutations particularly prevalent in pancreatic, colorectal, and lung adenocarcinomas. We chose the G12V mutation as the target because a relevant peptide containing it was predicted to bind with high affinity to the HLA-A2, which is the most common HLA allele in many ethnic groups (32). This in silico prediction was made using the NetMHC v3.4 algorithm (33-35). Additionally, the critical mutant residue (V at codon 12) was expected to be exposed on the surface of the HLA protein based on structural studies of other peptide HLA-complexes (36). The peptide KLVVVGAVGV (SEQ ID NO: 4), in which the valine residue (V) at position 8 represents the G12V mutation, was chemically synthesized by conventional means. Peptides corresponding to the product of a mutant allele will henceforth be termed “mutant peptides,” while peptides representing the product of a wt allele will be referred to as “wt peptides.” The mutant KRAS peptide was then folded into a complex (monomer) of HLA-A2 and beta-2-microglobulin [KRAS(G12V)-HLA-A2]. Two peptides corresponding to wt KRAS sequences were also synthesized and folded with HLA-A2 or HLA-A3 to form KRAS(WT)-HLA-A2 and KRAS(WT)-HLA-A3 monomers, respectively. Additional mutant KRAS monomers corresponding to other codon 12 mutations were also assembled. In most cases, monomers were biotinylated to facilitate purification and subsequent experimentation (see Materials and Methods).

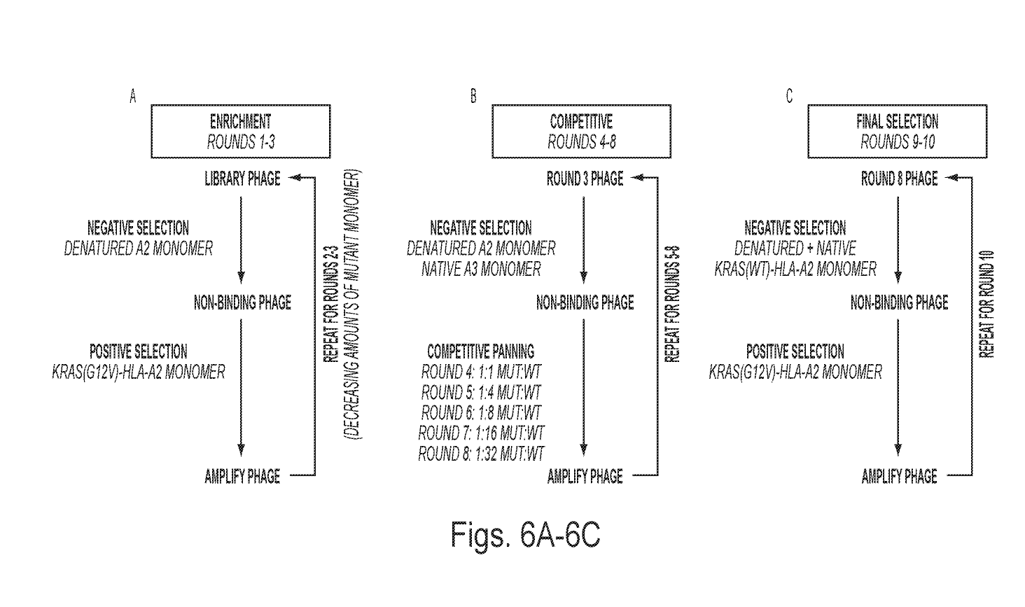

The phage display selection process consisted of 10 rounds of selection and amplification, which were divided into three distinct phases: an enrichment phase (rounds 1-3), a competitive phase (rounds 4-8), and a final selection phase (rounds 9-10) (FIG. 6). The overall objective of these phases was to maximize recovery of clones that bound KRAS(G12V)-HLA-A2 better than KRAS(WT)-HLA-A2 or HLA alone. In each round of the enrichment phase, negative selection with heat-denatured biotinylated HLA-A2 monomers was followed by positive selection with KRAS(G12V)-HLA-A2 (FIG. 6A and Materials and Methods). In each successive round (rounds 2 and 3), the amount of KRAS(G12V)-HLA-A2 monomer was reduced to enrich for stronger binders.

The novel competitive phase described in this study was intended to enrich for the rare mutant KRAS-(G12V)-HLA-A2 binders over KRAS(WT)-HLA-A2 binders and the much more frequent pan-HLA binders that we expected to be present in the library following the enrichment phase. Each round of the competitive phase began with negative selection using denatured HLA-A2 and native HLA-A3 monomers (FIG. 6B). Then, the phage were simultaneously incubated with KRAS(G12V)-HLA-A2 bound to streptavidin magnetic beads and KRAS(WT)-HLA-A2 bound to streptavidin agarose beads. KRAS(WT)-HLA-A2 served as a competitor, as phage bound to it would not be recovered in the magnetic bead trapping step (FIG. 6B). Moreover, in each round of the competitive phase, decreasing amounts of KRAS(G12V)-HLA-A2, but the same amount of KRAS(WT)-HLA-A2, were employed in an attempt to enrich for high affinity binders. In the final selection phase, each round started with stringent negative selection using a vast excess of denatured and native KRAS(WT)-HLA-A2 monomer and proceeded with positive selection with KRAS(G12V)-HLA-A2 monomer (FIG. 6C).

### EXAMPLE 4

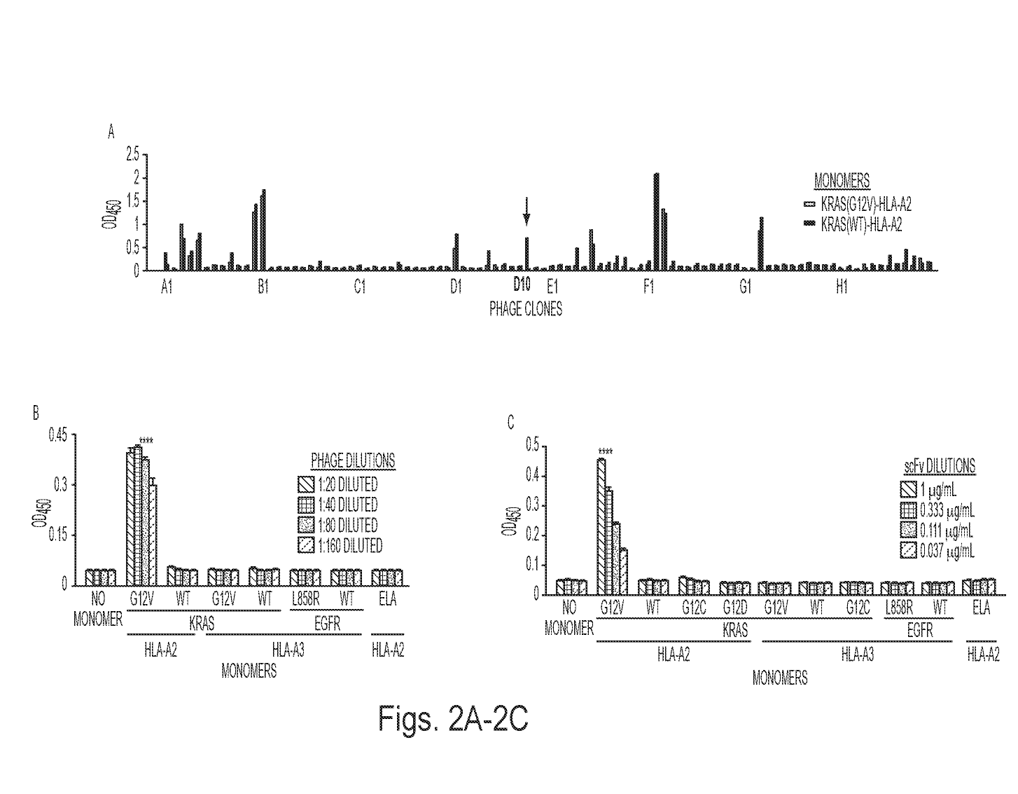

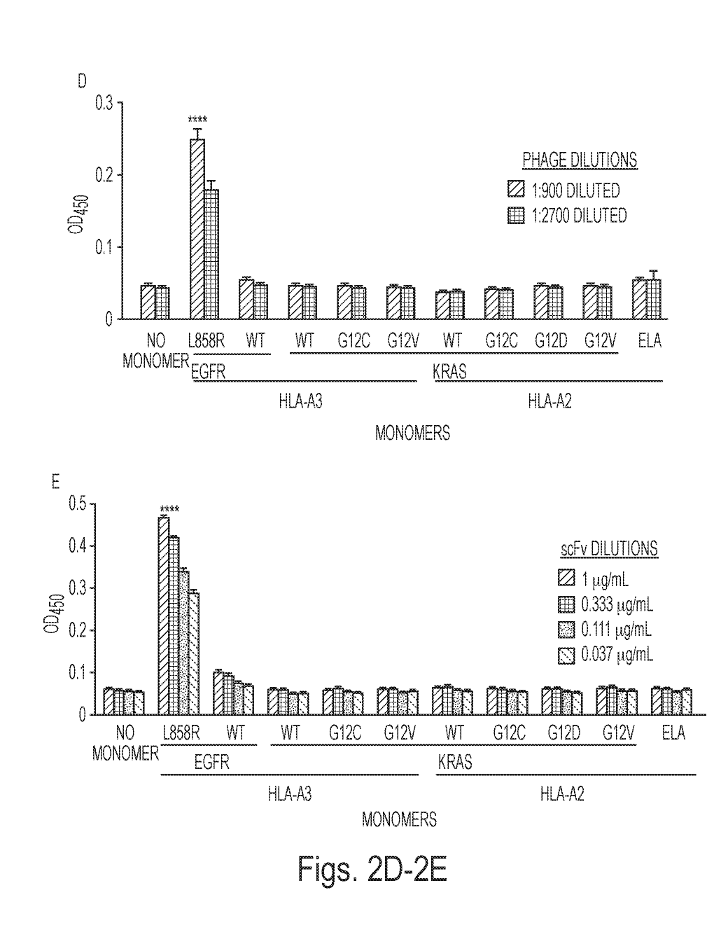

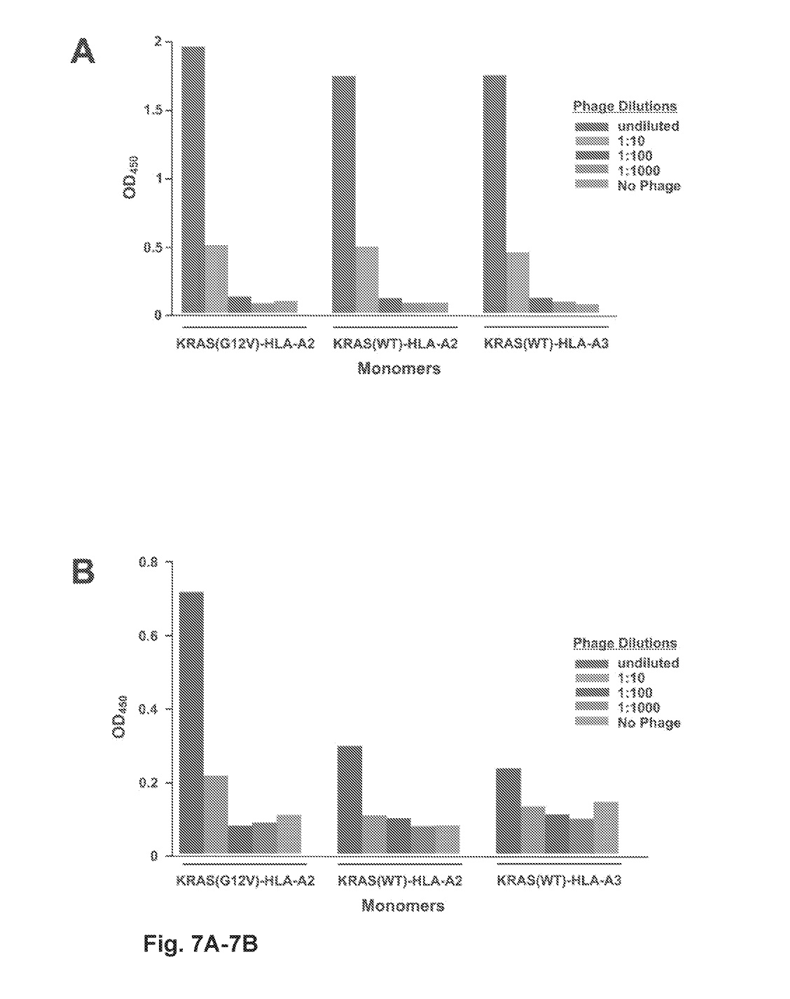

Evaluation of the selected phage clones. We used an enzyme-linked immunosorbant assay (ELISA) to evaluate the binding of phage to peptide-HLA complexes. After the enrichment phase (FIG. 6A), the selected phage (en masse) did not show preference for mutant over wt KRAS peptides complexed to HLA-A2, or preference for KRAS peptides bound to HLA-A2 over KRAS peptides bound to HLA-A3. (FIG. 7A.) Only after the final selection phase (FIG. 6C) did specificity for mutant KRAS bound to HLA-A2 emerge. In particular, these phage bound to KRAS(G12V)-HLA-A2 better than to KRAS(WT)-HLA-A2 or to KRAS(WT)-HLA-A3 (FIG. 7B). These phage were cloned by limiting dilution and expanded in a 96-well plate format. One clone (D10; SEQ ID NO: 37) showed substantial binding to the KRAS(G12V)-HLA-A2 monomer (FIG. 2A). The D10 clone was highly specific to KRAS(G12V)-HLA-A2, as it failed to bind all other monomers tested (FIG. 2B).

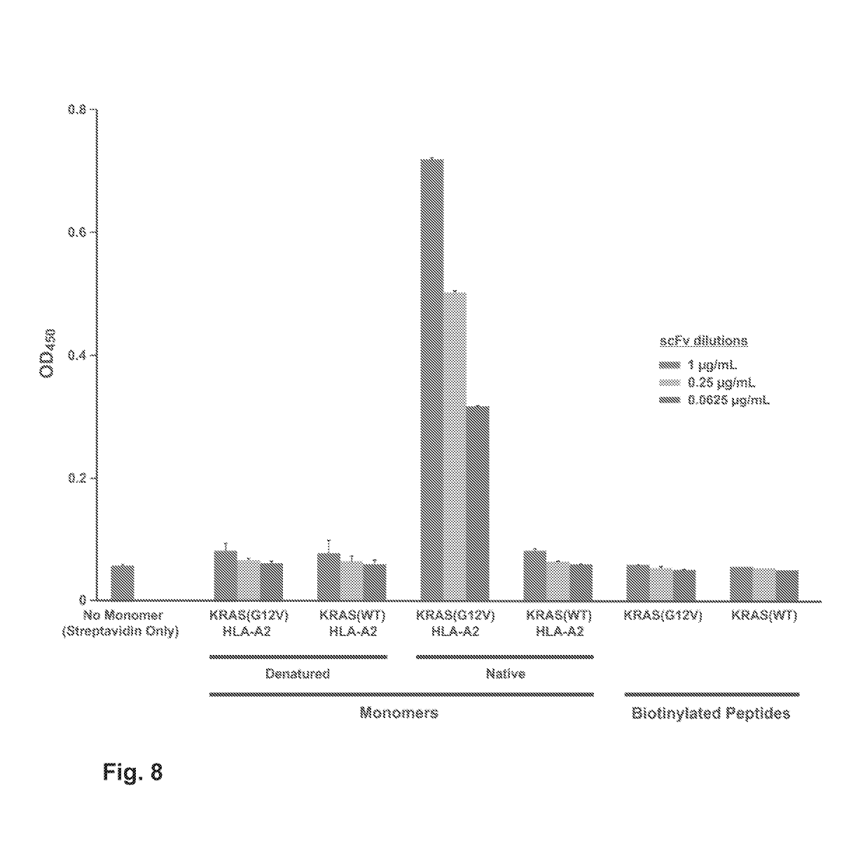

To produce D10 scFv uncoupled from M13 pIII, single-stranded DNA (ssDNA) from the D10 phage was purified. The scFv portion was amplified by polymerase chain reaction (PCR), sequenced, and cloned into a prokaryotic expression vector containing either a Flag or a V5 epitope tag in addition to a 6× His tag. This facilitated high-level expression and affinity purification of D10 scFv. Similar to the phage expressing D10 scFv:pIII fusion protein, purified D10 scFv interacted with KRAS(G12V)-HLA-A2 in a highly specific fashion (FIG. 2C). Importantly, the D10 scFv did not show any binding above background to KRAS(WT)-HLA-A2, KRAS(WT)-HLA-A3, or to other KRAS mutants (KRAS G12C or KRAS G12D) bound to HLA-A2. Additionally, D10 scFv did not bind to KRAS peptides when not complexed with HLA proteins (FIG. 8). These results demonstrate successful selection for scFv bound to peptides in the context of HLA.

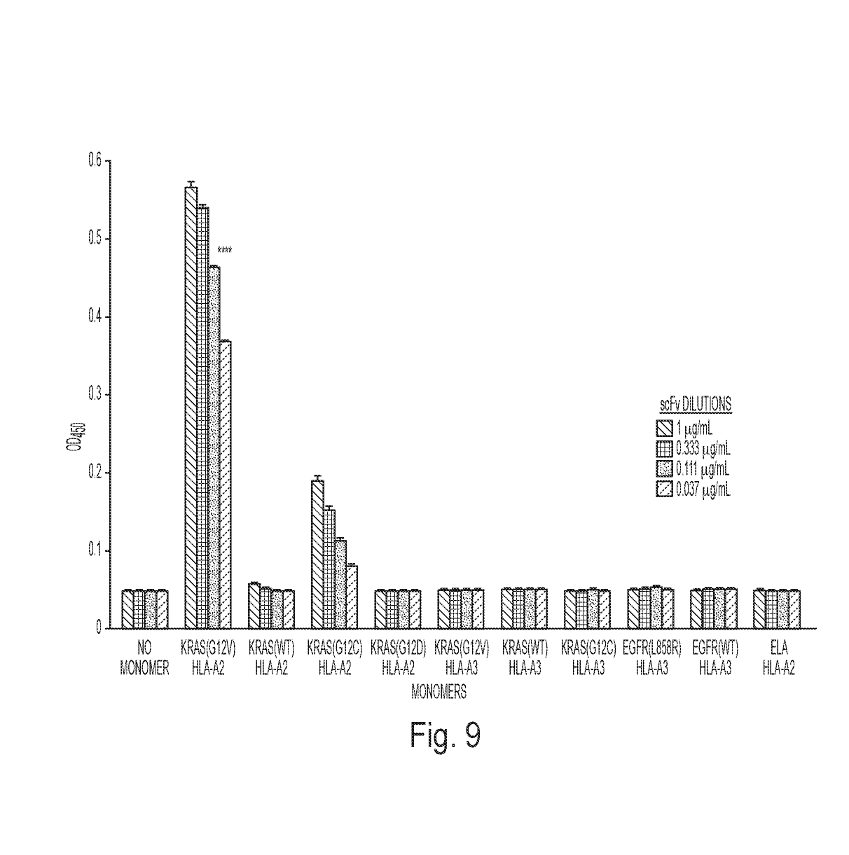

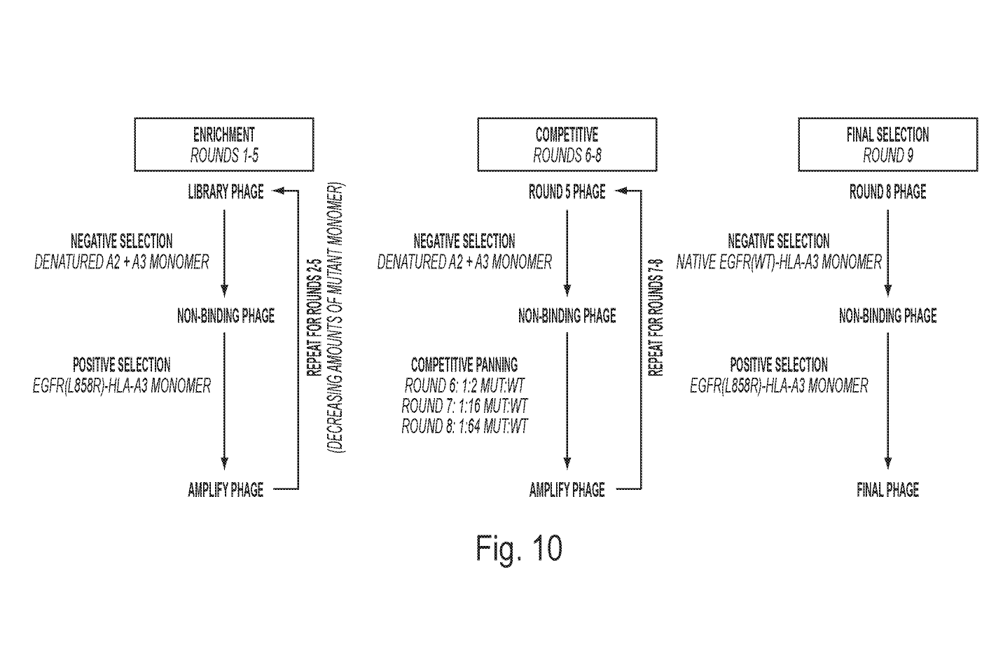

Affinity Maturation. The affinity of the D10 scFv for KRAS(G12V)-HLA-A2 was estimated to be 49 nM, using the AlphaScreen® method of affinity measurement (37). We next proceeded to affinity mature D10. Briefly, a library of D10 scFv mutagenized through error-prone PCR was generated from the original D10 scFv sequence and subject to three rounds of selection against the KRAS(G12V)-HLA-A2 and KRAS(WT)-HLA-A2 monomers. Evaluation of the clones yielded a candidate, D10-7, which showed a newly acquired capacity to bind to KRAS(G12C)-HLA-A2, while still retaining the ability to differentiate between mutant and wild type KRAS epitopes (FIG. 9). To compare D10-7 and D10 for their relative binding to KRAS(G12V)-HLA-A2, we used off-rate based assays to measure the koff value. Unlike affinity measurements, these assays allow for rapid comparison of multiple scFvs within the same test, thus providing a more direct comparison of the relative binding of multiple clones (38). The off-rate measurements showed an almost two-fold decrease in the disassociation rate constant for the affinity-matured D10-7 scFv (3.2×10-6 sec-1) as compared to the D10 scFv (5.7×10-6 sec-1). No measurable binding of the KRAS(WT)-HLA-A2 monomer to these scFvs occurred, documenting that the koff for KRAS(G12V)-HLA-A2 was at least 200-fold lower than for KRAS(WT)-HLA-A2. The large differential binding of these scFvs to mutant vs. wt peptides complexed with HLA-A2 was also evident in the other assays described below.

### EXAMPLE 5

Identification of phage that can bind to a different MANA. To determine whether this approach was applicable to other MANAs, we sought to identify scFvs specific for a mutant EGFR peptide complexed to different HLA allele. The EGFR L858R mutation is found in ˜10% of lung adenocarcinomas and accounts for ˜40% of all EGFR mutations in this cancer type (39). Codon 858 is in the cytoplasmic domain, rather than the extracellular or membrane domains, of the EGFR protein and normally should not be visible on the cell surface (40). A peptide (KITDFGRAK; SEQ ID NO: 9) containing this mutation was predicted to bind at high-affinity to the HLA-A3 allele when analyzed by the NetMHC v3.4 algorithm. To identify scFvs specific to this peptide-HLA complex, we adopted a modified scheme of selection and amplification in which decreasing concentrations of Isopropyl β-D-1-thiogalactopyranoside (IPTG) were used to reduce scFv expression in the later rounds. Additionally, the number of rounds in each selection phase was adjusted to favor the enrichment of desired phage rather than the elimination of undesired ones (FIG. 9 compared to FIG. 6, also see Materials and Methods). With these modifications, we were able to obtain a phage clone (C9; SEQ ID NO: 39) that showed selective binding to mutant EGFR peptide complexed to HLA-A3 [EGFR(L858R)-HLA-A3], compared to a variety of control monomers, including wt EGFR bound to HLA-A3 (FIG. 2D). The C9 scFv generated from this clone showed similar selective binding to EGFR(L858R)-HLA-A3 (FIG. 2E). The estimated k_{off} of the mutant EGFR peptide bound to HLA-A3 was an order of magnitude lower than the k_{off} of the wt peptide (value of 2.6×10⁻⁶ sec⁻¹ vs. 3.0×10⁻⁵ sec⁻¹, respectively).

### EXAMPLE 6

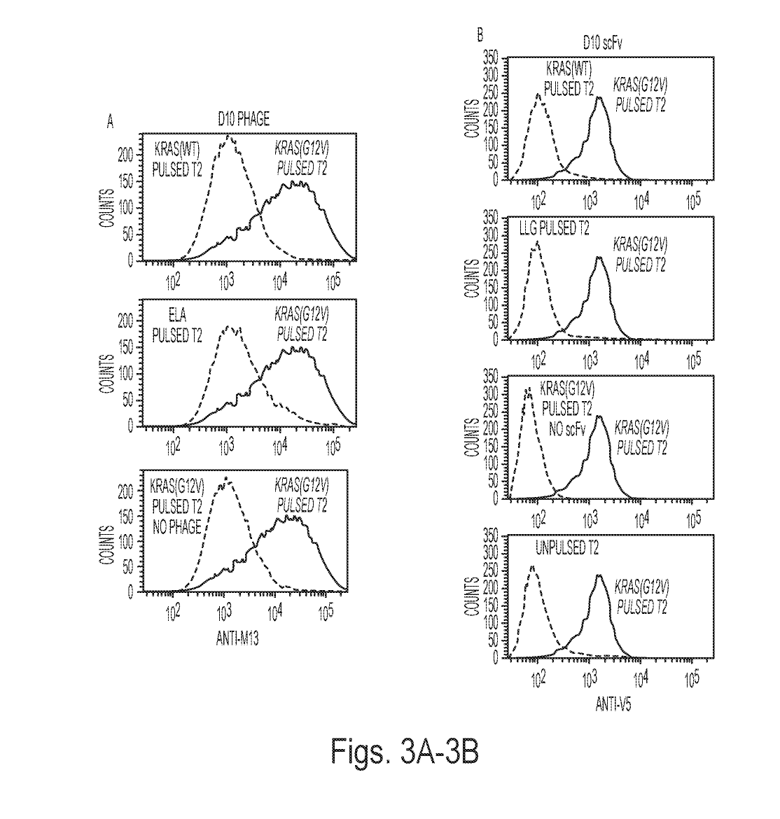

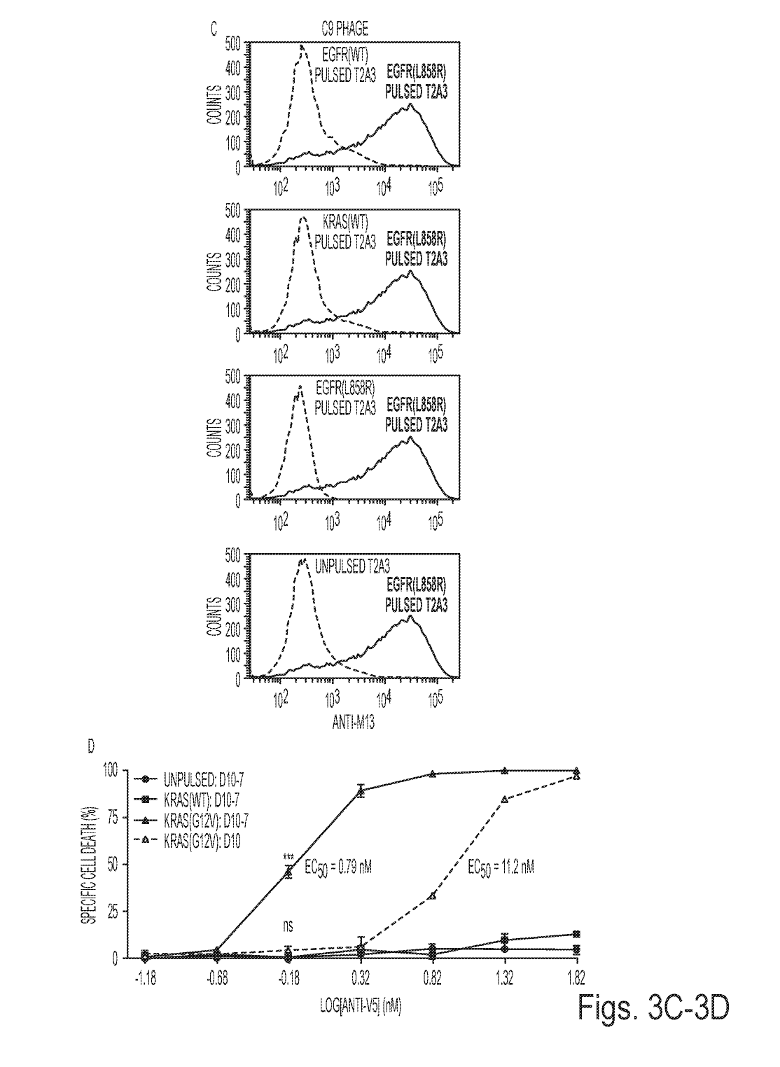

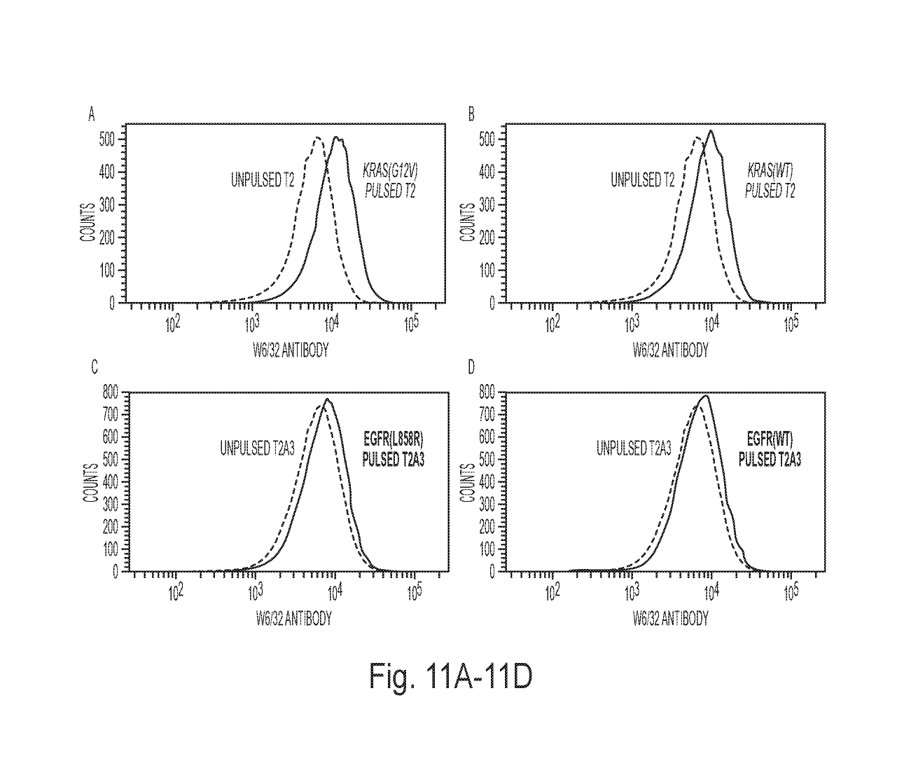

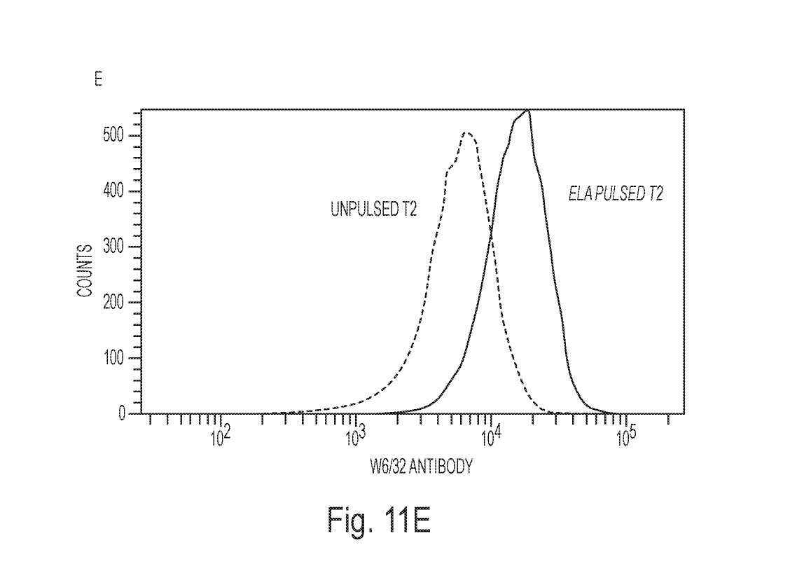

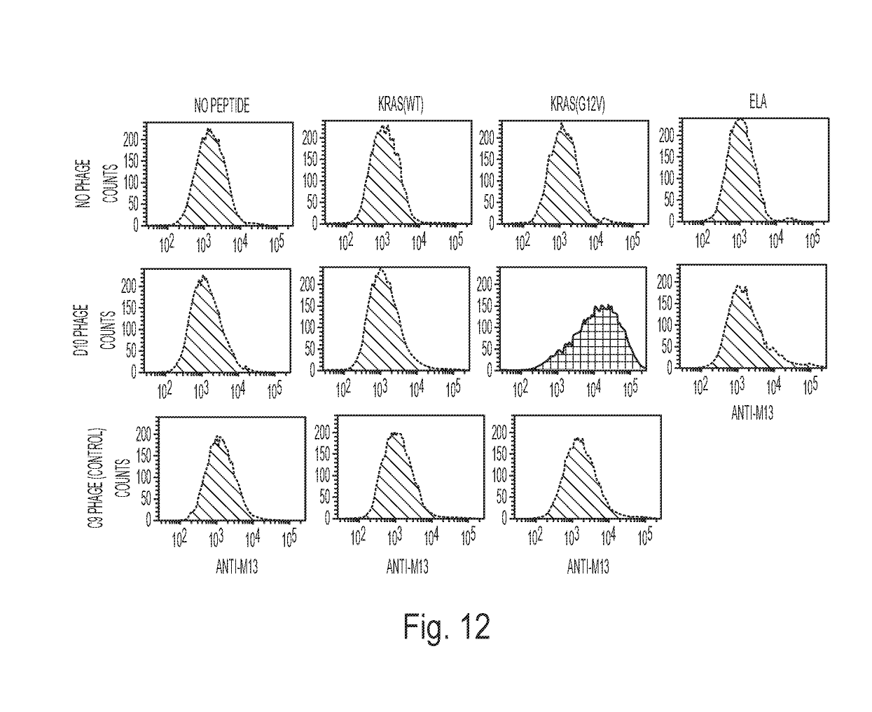

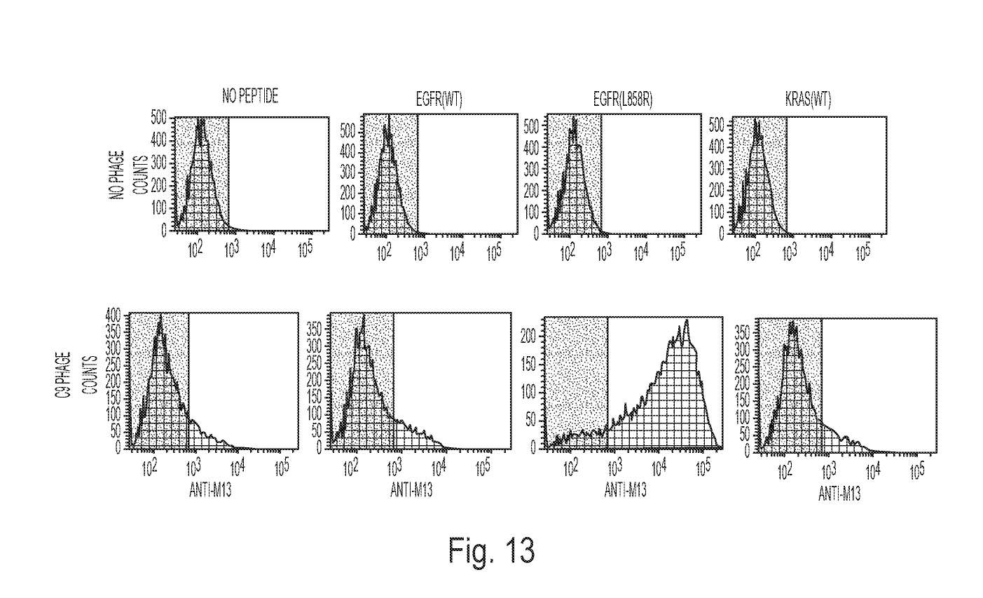

Selective binding to cells displaying mutant peptides on the cell surface. We next attempted to determine whether the D10 and C9 scFvs would bind to mutant KRAS and EGFR peptide-HLA complexes on the surface of cells. The T2 cell line was derived from an Epstein-Barr virus-transformed human lymphoblast line defective in presentation of endogenous HLA-associated peptide antigens due to a deletion that involves genes for TAP1 and TAP2 peptide transporters (41). T2A3 is a modified version of T2 with stable expression of the HLA-A3 transgene (42, 43). T2 and T2A3 cells express low levels of HLA that can be stabilized by addition of exogenous HLA-binding peptides, and thus can serve as a platform for assaying interactions with specific HLA-binding peptides (44, 45). We first pulsed T2 cells with KRAS(G12V), KRAS(WT), or a negative control peptide. To assess loading efficiency, we used the W6/32 antibody that targets HLA molecules stabilized by any HLA-binding peptides. The efficiency of peptide loading between wild type and mutant peptides were comparable as suggested by anti-W6/32 staining (FIG. 11). Analysis of the pulsed cells by flow cytometry after incubation with D10 phage showed that binding of the D10 phage to KRAS(G12V) peptide-pulsed cells was evident, while marginal binding (over background) to the KRAS(WT) or control peptide-pulsed cells was observed (FIG. 3A, FIG. S8). A similar experiment with purified D10 scFv rather than D10 phage confirmed the selective binding to KRAS(G12V) presented on the cell surface (FIG. 3B). We also pulsed T2A3 cells with mutant EGFR(L858R), EGFR(WT), or a negative control peptide, and assessed C9 phage binding by flow cytometry. Again, binding of the C9 phage to the EGFR(L858R) peptide-pulsed cells was evident, while no binding to the EGFR(WT) or control peptide-pulsed cells was observed (FIG. 3C, FIG. 13). Only background fluorescence was observed when phage or scFvs were not included in the reaction or the cells were not loaded with the peptides (FIG. 3A-3C, FIGS. 12 and 13).

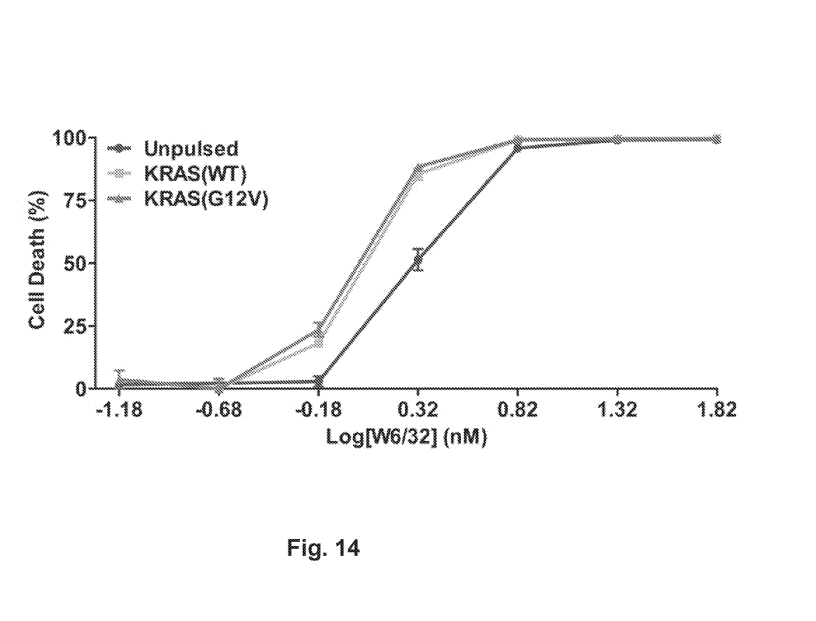

We next sought to determine whether T2 cells pulsed with the mutant KRAS(G12V) peptide could be targeted by D10 scFv and killed in a Complement-Dependent Cytotoxicity (CDC) assay. As a positive control, we pulsed T2 cells with KRAS(G12V) or KRAS(WT), peptides and performed a CDC assay with the W6/32 antibody. As expected, the antibody killed peptide-pulsed T2 cells efficiently in the presence of complement (FIG. 14). We then tested D10 scFv and the affinity-matured D10-7 scFv, both conjugated to an anti-V5 epitope tag antibody containing the complement-fixing Fc region in the CDC assay. Both scFvs killed the KRAS(G12V)-pulsed T2 cells in a dose-dependent fashion and the affinity-matured D10-7 scFv showed a remarkable improvement in killing efficiency (EC50 of 0.79 nM for D10-7 vs. EC50 of 11.2 nM for D10, FIG. 3D). In contrast, cells pulsed with KRAS(WT) or not pulsed with exogenous peptides showed only marginal cell death.

### EXAMPLE 7

Generation of a full-length MANAbody. Clinical applications of immunotherapeutic reagents generally employ complete antibodies, including the Fc domain, rather than just the scFv component. Another attribute of complete antibodies is the higher avidity achieved as a result of bivalency of the initially monovalent scFvs. To generate a complet MANAbody from the D10 scFv, we grafted the D10 scFv sequence onto the constant region of the clinically used humanized 4D5 antibody trastuzumab. High levels of expression of the D10 MANAbody were achieved in mammalian cells (139 milligrams of protein per liter of Expi293 cell culture). Similar to the D10 scFv, the D10 MANAbody interacted with KRAS(G12V)-HLA-A2, as assessed by ELISA (FIG. 4A). No binding was observed to the KRAS(WT)-HLA-A2 monomer or to any other monomer tested. The D10 MANAbody also showed relatively stronger staining of T2 cells pulsed with the mutant KRAS(G12V) peptide, compared to those pulsed with KRAS (WT) or a negative control peptide (FIG. 4B, FIG. 15). Finally, the observed half-life of the full-length D10 MANAbody was similar to that of its scFv derivative when assessed for its monovalent dissassociation. Thus, D10 MANAbody retained the high specificity and low dissociation rate constant observed with the D10 scFv.

### EXAMPLE 8

Modifications to phage panning protocols. Variations of the phage selection method previously described were performed to identify antibodies against additional HLA-peptide complexes (CTNNB1 S45F) and (EGFR T790M). CTNNB1 is the gene name coding for protein beta-catenin. These names are used interchangeably in this document.

The first change is the inclusion of cells from cell lines that express the particular HLA allele that is being screened for. However, these cells that do not contain the relevant mutation of interest. We add these cells to the negative selection step that occurs at the beginning of each round (the same step where we interrogate the phage against denatured HLA and naked streptavidin beads). The cells can be added to this step for the first two rounds or for the duration of screening. The purpose of this step is to remove any phage that bind to similar HLA-epitope sequences as our mutant epitope.

We also demonstrate that by altering the number of rounds of enrichment, competitive, and final selection phases we are able to identify antibodies had not been detected previously. In particular, 0 or 1 rounds of enrichment phase were performed, followed by up to 5 rounds of competitive selection. However, starting after the second round of competitive selection, aliquots of phage were shifted to negative selection at each day (this both shortens the number of rounds of phage selection and allows us to identify scFv candidates that were not present in later rounds of panning).

After 2, 3, and 4 rounds of competitive selection, phage were subjected to two consecutive rounds of negative selection, resulting in phage that had undergone 4 to 8 rounds of total selection.

These changes resulted in identifying scFv candidates against beta-catenin/CTNNB1 (S45F)-HLA-A3 and EGFR T790M-HLA-A2 mutations and potentially p53 mutations.

### EXAMPLE 9

KRAS G12V-HLA-A2 clone F10. Phage selection for the F10 clone was carried out as described for C9 phage selection, with the exception that mutant [KRAS(G12V)-HLA-A2] and wild type [KRAS(WT)-HLA-A2] monomers were used. This demonstrates that multiple scFv candidates can be identified for a given HLA complex.

The F10 clone:

(SEQ ID NO: 21)
DIQMTQSPSSLSASVGDRVTITCRASQDVNTAVAWYQQKPGKAPKLLIYS
ASFLYSGVPSRFSGSRSGTDFTLTISSLQPEDFATYYCQQYYYYPPTFGQ
GTKVEIKRTGGGSGGGGSGASEVQLVESGGGLVQPGGSLRLSCAASGFNI
NSNYIHWVRQAPGKGLEWVAYITPETGYYRYADSVKGRFTISADTSKNTA
YLQMNSLRAEDTAVYYCSRNYYSAYAMDVWGQGTLVTVSS

### EXAMPLE 10

Affinity maturation of KRAS F10 clone. As with KRAS D10 clone, the F10 scFv is also able to undergo effective affinity maturation and variants retain their specificity for KRAS mutant over KRAS wild type.

KRAS(wt)-HLA-A2 signal remains near background, as does the F10 scFv (this is due to the inclusion of an N-terminal epitope tag). F10 affinity matured variants display remarkable binding (as much as a 131-fold increase in mean fluorescence intensity).

scFv sequences for F10 Affinity Matured derivatives:

KRAS G12V_F10 AM#1
(SEQ ID NO: 22)
DIQMTQSPSSLSASVGDRVTITCRASQDVNTAVAWYQQKPGKAPKLLIYS
ASFLYSGVPSRFSGSRSGTDFTLTISSLQPEDFATYYCQQYYYYPPTFGQ
GTKVEIKRTGGGSGGGGSGASEVQLVESGGGLVQPGGSLRLSCAASGFNI
NSNYIHWVRQAPGKGLEWVAYITPETGYYHYADSVKGRFTISADTSKNTA
YLQMNSLRAEDTAVYYCSRNYYSAYAMDVWGQGTLVTVSS
KRAS G12V_F10 AM#2
(SEQ ID NO: 23)
DIQMTQSPSSLSASVGDRVTITCRASQDVNTAVAWYQQKPGKAPKLLIYG
ASFLYSGVPSRFSGSRSGTDFTLTISSLQPEDFATYYCQQYYYYPPTFGQ
GTKVEIKRTGGGSGGGGSGASEVQLVESGGGLVQPGGSLRLSCAASGFYI
NSNYIHWVRQAPGKGLEWVAYITPETGYYHYADSVKGRFTISADTSKNTA
YLQMNSLRAEDTAVYYCSRNYYSAYAMDVWGQGTLVTVSS
KRAS G12V_F10 AM#3
(SEQ ID NO: 24)
DIQMSQSPSSLSASVGDRVTITCRTSQDANTAVAWYQQKPGKAPKLLFYS
ASFLFSGVPSRFSGSRSGTDFTLTISSLQPEDFATYYCQQYYYYPPTFGQ
GTKVEIKRTGGGSGGGGSGASEVQLVESGGGLVQPGGSLRLSCAASGFNI
NSNYIHWVRQAPGKGLEWVAYITPETGYYRYADSVKGRFTISADTSKNTA
YLQMNSLRAEDTAVYYCSRNYYSAYAMDVWGQGTLVTVSS

### EXAMPLE 11

CTNNB1 (S45F)-HLA-A3 oncogenic mutation (TTAPFLSGK; SEQ ID NO: 27). Mutations at residue 45 of the protein product of CTNNB1 (beta-catenin) are the second most common in the oncogene. (The S→F mutation is the most prevalent Amino acid change). Identifying an antibody against S45F also suggests that antibody derivatives against S45P are possible. Additionally the T41A mutation, which is the most common CTNNB1 mutation, is also predicted to bind HLA-A3 with the same amino acid coordinates (ATAPSLSGK; SEQ ID NO: 36). This demonstrates the feasibility of targeting this mutation as well.

Changes to panning for beta-catenin/CTNNB1 S4F5 and EGFR T790M mutations are described below. This demonstrates the flexibility inherent in the competition based screening.

The E10 scFv sequence:

(SEQ ID NO: 16)
DIQMTQSPSSLSASVGDRVTITCRASQDVNTAVAWYQQKPGKAPKLLIYS
ASFLYSGVPSRFSGSRSGTDFTLTISSLQPEDFATYYCQQSYYSPPTFGQ
GTKVEIKRTGGGSGGGASEVQLVESGGGLVQPGGSLRLSCAASGFNINNT
YIHWVRQAPGKGLEWVASIYPTDGYTRYADSVKGRFTISADTSKNTAYLQ
MNSLRAEDTAVYYCSRTYYSYYSAMDVWGQGTLVTVSS

W6/32 data shows an increase in antibody binding over b2m (negative control) showing that the peptide is presented by HLA-A3 complexes. E10 phage staining shows that the scFv binds specifically to S45F epitopes (80,400 k MFIs) over control peptides (600-800 MFI).

Phage clone E10 is specific to CTNNB S45F peptide-pulsed cells.

### EXAMPLE 12

EGFR T790M. The EGFR T790M mutation is the second most frequent EGFR mutation (after L858R). This is a significant mutation because it appears frequently in response to anti-EGFR therapies as a resistance mutation. Additionally evidence in the literature suggests that the T790M mutation is endogenously processed and presented on tumor cells by HLA-A2 complexes (as both 9 and 10 amino acid epitopes).

The 9 amino acid mutant epitope is:

IMQLMPFGC (SEQ ID NO: 28; mutant residue T→M is underlined and bolded)

The 10 amino acid mutant epitope is:

LIMQLMPFGC (SEQ ID NO: 29; mutant residue T->M is underlined and bolded)

### EXAMPLE 13

Phage Selection. Phage selection was done as described above. Potential candidates (with different scFv sequences) from two and three and four days of competitive selection (followed by two days of negative selection) were identified. These candidates are referred to as D2D6, D3E6, D2D8. D3E6 looked the most promising among this cohort.

The D3E6 scFv sequence is:

(SEQ ID NO: 18)
DIQMTQSPSSLSASVGDRVTITCRASQDVNTAVAWYQQKPGKAPKLLIYS
ASFLYSGVPSRFSGSRSGTDFTLTISSLQPEDFATYYCQQYYYYPPTFGQ
GTKVEIKRTGGGSGGGGSGGGASEVQLVESGGGLVQPGGSLRLSCAASGF
NISTSYIHWVRQAPGKGLEWVATIDPNDGYSRYADSVKGRFTISADTSKN
TAYLQMNSLRAEDTAVYYCSRTNNTAADAMDVWGQGTLVTVSS

The D2D6 scFv sequence is:

(SEQ ID NO: 40)
DIQMTQSPSSLSASVGDRVTITCRASQDVNTAVAWYQQKPGKAPKLLIYS
ASFLYSGVPSRFSGSRSGTDFTLTISSLQPEDFATYYCQQSYYSPPTFGQ
GTKVEIKRTGGGSGGGGSGGGASEVQLVESGGGLVQPGGSLRLSCAASGF
NITSSYIHWVRQAPGKGLEWVAYISPADGYNRYADSVKGRFTISADTSKN
TAYLQMNSLRAEDTAVYYCSRTDSTAYTAMDVWGQGTLVTVSS

The D2D8 scFv sequence is:

(SEQ ID NO: 41)
DIQMTQSPSSLSASVGDRVTITCRASQDVNTAVAWYQQKPGKAPKLLIYS
ASFLYSGVPSRFSGSRSGTDFTLTISSLQPEDFATYYCQQYYSYPPTFGQ
GTKVEIKRTGGGSGGGGSGGGASEVQLVESGGGLVQPGGSLRLSCAASGF
NINSSYIHWVRQAPGKGLEWVAYISPTDGYYRYADSVKGRFTISADTSKN
TAYLQMNSLRAEDTAVYYCSRTSDTSYAAMDVWGQGTLVTVSS

### EXAMPLE 14

p53 R248W Clones. TP53 is the most commonly mutated gene in cancer. We have obtained scFVs capable of binding to the p53 R248W mutation. We are currently testing their specificity, but demonstrate that antibodies against this epitope may be obtained. Another common mutation p53 R248Q, which is identical in sequence except for the W to Q change, binds in a similar fashion to HLA-A2.

Clone D2F2 sequence:

(SEQ ID NO: 25)
DIQMTQSPSSLSASVGDRVTITCRASQDVNTAVAWYQQKPGKAPKLLIYS
ASFLYSGVPSRFSGSRSGTDFTLTISSLQPEDFATYYCQQSYSSPPTFGQ
GTKVEIKRTGGGSGGGASEVQLVESGGGLVQPGGSLRLSCAASGFNINDT
YIHWVRQAPGKGLEWVAYISPASGNSRYADSVKGRFTISADTSKNTAYLQ
MNSLRAEDTAVYYCSRSYAAMDVWGQGTLVTVSS

### EXAMPLE 15

ABL1 E255K mutation (KVYEGVWKK; SEQ ID NO: 26). ABL1 is mutated in ˜30% of all CMLs. E255K mutation confers drug resistance to imatinab and nilotinab. The mutation is predicted to reside at position 1 within the epitope. This makes it very difficult for an antibody or TCR to distinguish between mutant and wild type. However, the predicted affinity of HLA-A3 for the mutant epitope is 10-fold higher than the predicted wild type affinity (29 nM vs. 228 nM). Additionally, proteolytic processing of epitopes with different N-terminal amino acids may results in different cleavage products, thus affecting endogenous presentation. This suggests that scFv recognition of both mutant and wild type epitopes (with mutations at position 1) may not hinder in vivo mutant epitope specificity.

Abl1 E255K_hit 1
(SEQ ID NO: 14)
MASDIQMTQSPSSLSASVGDRVTITCRASQDVNTAVAWYQQKPGKAPKLL
IYSASFLYSGVPSRFSGSRSGTDFTLTISSLQPEDFATYYCQQYYSSPPT
FGQGTKVEIKRTGGGSGGGGSGGGASEVQLVESGGGLVQPGGSLRLSCAA
SGFNINSSSIHWVRQAPGKGLEWVASIAPARGSTRYADSVKGRFTISADT
SKNTAYLQMNSLRAEDTAVYYCSRNYYAYTAMDVWGQGTLVTVSS
Abl1 E255K_hit 2
(SEQ ID NO: 15)
DIQMTQSPSSLSASVGDRVTITCRASQDVNTAVAWYQQKPGKAPKLLIYS
ASFLYSGVPSRFSGSRSGTDFTLTISSLQPEDFATYYCQQYSSSPPTFGQ
GTKVEIKRTGGGSGGGASEVQLVESGGGLVQPGGSLRLSCAASGFNINTS
YIHWVRQAPGKGLEWVASIYPNDGYNRYADSVKGRFTISADTSKNTAYLQ
MNSLRAEDTAVYYCSRAAYAMDVWGQGTLVTVSS

### REFERENCES

The disclosure of each reference cited is expressly incorporated herein.
1. Fearon E R & Vogelstein B (1990) A genetic model for colorectal tumorigenesis. Cell 61(5):759-767.
2. Vogelstein B, et al. (2013) Cancer genome landscapes. Science 339(6127):1546-1558.
3. Tsai J, et al. (2008) Discovery of a selective inhibitor of oncogenic B-Raf kinase with potent antimelanoma activity. Proc Natl Acad Sci USA 105(8):3041-3046.
4. Halilovic E & Solit D B (2008) Therapeutic strategies for inhibiting oncogenic BRAF signaling. Curr Opin Pharmacol 8(4):419-426.
5. Bradbury A R, Sidhu S, Dubel S, & McCafferty J (2011) Beyond natural antibodies: the power of in vitro display technologies. Nat Biotechnol 29(3):245-254.
6. Sliwkowski M X & Mellman I (2013) Antibody therapeutics in cancer. Science 341(6151):1192-1198.
7. Weiner L M, Surana R, & Wang S (2010) Monoclonal antibodies: versatile platforms for cancer immunotherapy. Nature reviews. Immunology 10(5):317-327.
8. Ayriss J, Woods T, Bradbury A, & Pavlik P (2007) High-throughput screening of single-chain antibodies using multiplexed flow cytometry. J Proteome Res 6(3):1072-1082.
9. Kato Y, et al. (2009) A monoclonal antibody IMab-1 specifically recognizes IDH1R132H, the most common glioma-derived mutation. Biochem Biophys Res Commun 390(3):547-551.
10. Yu J, et al. (2009) Mutation-specific antibodies for the detection of EGFR mutations in non-small-cell lung cancer. Clin Cancer Res 15(9):3023-3028.
11. Roblek M, et al. (2010) Monoclonal antibodies specific for disease-associated point-mutants: lamin A/C R453W and R482W. PLoS One 5(5):e10604.
12. Segal N H, et al. (2008) Epitope landscape in breast and colorectal cancer. Cancer Res 68(3):889-892.
13. Rahma O E, et al. (2014) The immunological and clinical effects of mutated ras peptide vaccine in combination with IL-2, GM-CSF, or both in patients with solid tumors. Journal of translational medicine 12:55.
14. Abrams S I, Hand P H, Tsang K Y, & Schlom J (1996) Mutant ras epitopes as targets for cancer vaccines. Semin Oncol 23(1):118-134.
15. Gubin M M, et al. (2014) Checkpoint blockade cancer immunotherapy targets tumour-specific mutant antigens. Nature 515(7528):577-581.
16. Castle J C, et al. (2012) Exploiting the mutanome for tumor vaccination. Cancer Res 72(5):1081-1091.
17. Vonderheide R H & June C H (2014) Engineering T cells for cancer: our synthetic future. Immunol Rev 257(1):7-13.
18. Schreiber R D, Old L J, & Smyth M J (2011) Cancer immunoediting: integrating immunity's roles in cancer suppression and promotion. Science 331(6024):1565-1570.
19. Robbins P F, et al. (2013) Mining exomic sequencing data to identify mutated antigens recognized by adoptively transferred tumor-reactive T cells. Nat Med 19(6):747-752.
20. Yadav M, et al. (2014) Predicting immunogenic tumour mutations by combining mass spectrometry and exome sequencing. Nature 515(7528):572-576.
21. Bondgaard A L, Hogdall E, Mellemgaard A, & Skov B G (2014) High specificity but low sensitivity of mutation-specific antibodies against EGFR mutations in non-small-cell lung cancer. Mod Pathol 27(12):1590-1598.
22. Sidhu S S, et al. (2004) Phage-displayed antibody libraries of synthetic heavy chain complementarity determining regions. J Mol Biol 338(2):299-310.
23. Carter P, et al. (1992) Humanization of an anti-p185HER2 antibody for human cancer therapy. Proc Natl Acad Sci USA 89(10):4285-4289.
24. Bostrom J & Fuh G (2009) Design and construction of synthetic phage-displayed Fab libraries. Methods in molecular biology 562:17-35.
25. Eigenbrot C, Randal M, Presta L, Carter P, & Kossiakoff A A (1993) X-ray structures of the antigen-binding domains from three variants of humanized anti-p185HER2 antibody 4D5 and comparison with molecular modeling. J Mol Biol 229(4):969-995.
26. Fellouse F A, et al. (2007) High-throughput generation of synthetic antibodies from highly functional minimalist phage-displayed libraries. J Mol Biol 373(4):924-940.
27. Koide S & Sidhu S S (2009) The importance of being tyrosine: lessons in molecular recognition from minimalist synthetic binding proteins. ACS chemical biology 4(5):325-334.
28. Kuroda D, Shirai H, Kobori M, & Nakamura H (2008) Structural classification of CDR-H3 revisited: a lesson in antibody modeling. Proteins 73(3):608-620.
29. Kunik V, Peters B, & Ofran Y (2012) Structural consensus among antibodies defines the antigen binding site. PLoS computational biology 8(2):e1002388.
30. Sela-Culang I, Kunik V, & Ofran Y (2013) The structural basis of antibody-antigen recognition. Frontiers in immunology 4:302.
31. Kinde I, Wu J, Papadopoulos N, Kinzler K W, & Vogelstein B (2011) Detection and quantification of rare mutations with massively parallel sequencing. Proc Natl Acad Sci USA 108(23):9530-9535.
32. Ellis J M, et al. (2000) Frequencies of HLA-A2 alleles in five U.S. population groups. Predominance Of A*02011 and identification of HLA-A*0231. Human immunology 61(3):334-340.
33. Nielsen M, et al. (2003) Reliable prediction of T-cell epitopes using neural networks with novel sequence representations. Protein science: a publication of the Protein Society 12(5):1007-1017.
34. Lundegaard C, et al. (2008) NetMHC-3.0: accurate web accessible predictions of human, mouse and monkey MHC class I affinities for peptides of length 8-11. Nucleic Acids Res 36(Web Server issue):W509-512.
35. Lundegaard C, Lund O, & Nielsen M (2008) Accurate approximation method for prediction of class I MHC affinities for peptides of length 8, 10 and 11 using prediction tools trained on 9mers. Bioinformatics 24(11):1397-1398.
36. Bouvier M, Guo H C, Smith K J, & Wiley D C (1998) Crystal structures of HLA-A*0201 complexed with antigenic peptides with either the amino- or carboxyl-terminal group substituted by a methyl group. Proteins 33(1):97-106.
37. Miller, M. D., et al. (2005). “A human monoclonal antibody neutralizes diverse HIV-1 isolates by binding a critical gp41 epitope.” Proc Natl Acad Sci USA 102(41): 14759-14764.
38. Ylera, F., et al. (2013). “Off-rate screening for selection of high-affinity anti-drug antibodies.” Anal Biochem 441(2): 208-213.
39. D'Angelo S P, et al. (2011) Incidence of EGFR exon 19 deletions and L858R in tumor specimens from men and cigarette smokers with lung adenocarcinomas. J Clin Oncol 29(15):2066-2070.
40. Sharma S V, Bell D W, Settleman J, & Haber D A (2007) Epidermal growth factor receptor mutations in lung cancer. Nat Rev Cancer 7(3):169-181.
41. Salter R D, Howell D N, & Cresswell P (1985) Genes regulating HLA class I antigen expression in T-B lymphoblast hybrids. Immunogenetics 21(3):235-246.
42. Anderson K S, Alexander J, Wei M, & Cresswell P (1993) Intracellular transport of class I MHC molecules in antigen processing mutant cell lines. J Immunol 151(7):3407-3419.
43. Thomas A M, et al. (2004) Mesothelin-specific CD8(+) T cell responses provide evidence of in vivo cross-priming by antigen-presenting cells in vaccinated pancreatic cancer patients. J Exp Med 200(3):297-306.
44. Luft T, et al. (2001) Exogenous peptides presented by transporter associated with antigen processing (TAP)-deficient and TAP-competent cells: intracellular loading and kinetics of presentation. J Immunol 167(5):2529-2537.
45. Brickner A G, et al. (2006) The PANE1 gene encodes a novel human minor histocompatibility antigen that is selectively expressed in B-lymphoid cells and B-CLL. Blood 107(9):3779-3786.
46. Steinwand M, et al. (2014) The influence of antibody fragment format on phage display based affinity maturation of IgG. mAbs 6(1):204-218.
47. McConnell A D, et al. (2013) An integrated approach to extreme thermostabilization and affinity maturation of an antibody. Protein engineering, design & selection: PEDS 26(2):151-164.
48. Miller K R, et al. (2012) T cell receptor-like recognition of tumor in vivo by synthetic antibody fragment. PLoS One 7(8):e43746.
49. Verma B, et al. (2010) TCR mimic monoclonal antibody targets a specific peptide/HLA class I complex and significantly impedes tumor growth in vivo using breast cancer models. J Immunol 184(4):2156-2165.
50. Bernal M, et al. (2012) Implication of the β2-microglobulin gene in the generation of tumor escape phenotypes. Cancer Immunol Immunother 61(9):1359-71.
51. Nolan J P, Lauer S, Prossnitz E R, & Sklar L A (1999) Flow cytometry: a versatile tool for all phases of drug discovery. Drug Discov Today 4(4):173-180.
52. Porgador A, Yewdell J W, Deng Y, Bennink J R, & Germain R N (1997) Localization, quantitation, and in situ detection of specific peptide-MHC class I complexes using a monoclonal antibody. Immunity 6(6):715-726.
53. Hoof I, van Baarle D, Hildebrand W H, & Kesmir C (2012) Proteome sampling by the HLA class I antigen processing pathway. PLoS computational biology 8(5):e1002517.
54. Wang W, et al. (1997) A naturally processed peptide presented by HLA-A*0201 is expressed at low abundance and recognized by an alloreactive CD8+ cytotoxic T cell with apparent high affinity. J Immunol 158(12):5797-5804.
55. de Verteuil D, Granados D P, Thibault P, & Perreault C (2012) Origin and plasticity of MHC I-associated self peptides. Autoimmunity reviews 11(9):627-635.
56. Stone J D, Aggen D H, Schietinger A, Schreiber H, & Kranz D M (2012) A sensitivity scale for targeting T cells with chimeric antigen receptors (CARs) and bispecific T-cell Engagers (BiTEs). Oncoimmunology 1(6):863-873.
57. Jena B, Dotti G, & Cooper L J (2010) Redirecting T-cell specificity by introducing a tumor-specific chimeric antigen receptor. Blood 116(7):1035-1044.
58. Kershaw M H, Westwood J A, Slaney C Y, & Darcy P K (2014) Clinical application of genetically modified T cells in cancer therapy. Clinical & translational immunology 3(5):e16.
59. Link B K, et al. (1998) Anti-CD3-based bispecific antibody designed for therapy of human B-cell malignancy can induce T-cell activation by antigen-dependent and antigen-independent mechanisms. Int J Cancer 77(2):251-256.
60. Thakur A & Lum L G (2010) Cancer therapy with bispecific antibodies: Clinical experience. Current opinion in molecular therapeutics 12(3):340-349.

We claim:

1. An isolated molecule comprising an antibody variable region which specifically binds to a complex of a human leukocyte antigen (HLA) molecule and a peptide which is a portion of a protein,
wherein the peptide comprises a mutant residue, and wherein the mutant residue is in an intracellular epitope of the protein,
wherein the molecule does not specifically bind to the HLA molecule when the HLA molecule is not in said complex, and
wherein the molecule does not specifically bind to the peptide in its wild-type form.

2. The isolated molecule of claim 1 wherein said complex further comprises a β-2-microglobulin molecule.

3. The isolated molecule comprising an antibody variable region of claim 1 which is an scFv.

4. The isolated molecule comprising an antibody variable region of claim 1 which is a Fab.

5. The isolated molecule comprising an antibody variable region of claim 1 wherein the protein is an oncogenic protein.

6. The isolated molecule comprising an antibody variable region of claim 5 wherein the oncogenic protein is epidermal growth factor receptor (EGFR).

7. The isolated molecule comprising an antibody variable region of claim 6 wherein the oncogenic protein has a L858R mutation.

8. The isolated molecule comprising an antibody variable region of claim 6 wherein the oncogenic protein has a T790M mutation.

9. The isolated molecule comprising an antibody variable region of claim 5 wherein the oncogenic protein is ABL.

10. The isolated molecule comprising an antibody variable region of claim 5 wherein the oncogenic protein is a bcr/ABL fusion protein.

11. The isolated molecule comprising an antibody variable region of claim 9 wherein the oncogenic protein has an E225K mutation.

12. The isolated molecule comprising an antibody variable region of claim 5 wherein the oncogenic protein is beta-catenin.

13. The isolated molecule comprising an antibody variable region of claim 12 wherein the oncogenic protein has a S45F mutation.

14. The isolated molecule comprising an antibody variable region of claim 5 wherein the oncogenic protein is P53.

15. The isolated molecule comprising an antibody variable region of claim 14 wherein the oncogenic protein has a R248W mutation.

16. The isolated molecule comprising an antibody variable region of claim 14 wherein the oncogenic protein has a R248Q mutation.

17. The isolated molecule comprising an antibody variable region of claim 5 wherein the oncogenic protein is KRAS.

18. The isolated molecule comprising an antibody variable region of claim 17 wherein the oncogenic protein has a G12 mutation.

19. The isolated molecule comprising an antibody variable region of claim 17 wherein the oncogenic protein has a G12V mutation.

20. The isolated molecule comprising an antibody variable region of claim 17 wherein the oncogenic protein has a G12C mutation.

21. The isolated molecule comprising an antibody variable region of claim 17 wherein the oncogenic protein has a G12D mutation.

22. The isolated molecule comprising an antibody variable region of claim 1 wherein the protein is a tumor suppressor.

23. The isolated molecule comprising an antibody variable region of claim 1 which does not bind to the peptide when it is not in the complex.

24. The isolated molecule comprising an antibody variable region of claim 2 wherein the HLA molecule is HLA-A2.

25. The isolated molecule comprising an antibody variable region of claim 2 wherein the HLA molecule is HLA-A3.

26. The isolated molecule comprising an antibody variable region of claim 1 which is bound to a detectable label.

27. The isolated molecule comprising an antibody variable region of claim 1 which is bound to a therapeutic agent.

28. The isolated molecule comprising an antibody variable region of claim 1 which is expressed as part of a chimeric protein which comprises a transmembrane region and an intracellular domain to form a chimeric antigen receptor (CAR).

29. The isolated molecule comprising an antibody variable region of claim 1 which is expressed as part of a chimeric protein which comprises an scFv which specifically binds to CD3.

30. A method of selecting from a nucleic acid library an scFv or Fab or T cell receptor that specifically binds to a complex of a human leukocyte antigen (HLA) molecule and a first form of a peptide portion of a protein, wherein the first form comprises a mutant residue, and wherein the mutant residue is in an intracellular epitope of the protein, wherein the scFv or Fab or T cell receptor does not specifically bind to the HLA molecule when the HLA molecule is not in said complex, and wherein the scFv or Fab or TCR does not specifically bind to the peptide in its wild-type form, the method comprising a step of:
positively selecting for scFv or Fab or T cell receptors that bind to said complex in the presence of a competitor complex that comprises a second form of the peptide portion bound to HLA and β-2-microglobulin, wherein the second form is selected from the group consisting of a wild-type form and a peptide with a different mutant residue than the first form.

31. The method of claim 30 wherein said complex further comprises a β-2-microglobulin molecule.

32. The method of claim 31 which comprises the steps of:
a. negatively selecting for scFv or Fab or T cell receptors that bind to unfolded human leukocyte antigen (HLA);
b. positively selecting for scFv or Fab or T cell receptors that bind to a complex of HLA, β-2-microbglobulin and the peptide;
c. positively selecting for scFv or Fab or T cell receptors that bind to the complex in the presence of a competitor complex that comprises wild-type form of the peptide bound to HLA and β-2-microglobulin;
d. negatively selecting for scFv or Fab or T cell receptors that bind to HLA monomers containing wild type peptide;
e. positively selecting for scFv or Fab or T cell receptors that bind to the complex.

33. The method of claim 32 wherein pairs of steps (a) and (b), (a) and (c), and (d) and (e) are performed a plurality of times.

34. The method of claim 33 wherein after each pair of positive and negative selection steps, remaining scFv or Fab or T cell receptors are amplified.

35. The method of claim 33 wherein during successive performance of step (c), amounts of said complex and competitor complex are varied so that ratio of competitor complex to said complex increases.

36. The method of claim 30 wherein the library comprises a synthetic library.

37. The method of claim 30 wherein the library comprises a synthetic oligonucleotide library.

38. The method of claim 30 wherein the library is a phage display library.

39. The method of claim 30 wherein the library is a ribosome display library.

40. The method of claim 30 wherein the library is a yeast display library.

41. The method of claim 29 wherein the complex used for positively selecting is displayed on the surface of a cell.

42. A method of treating a subject with a cancer or with a resected tumor, comprising:
administering to the subject the isolated molecule of claim 27.

43. A method of detecting cancer cells in a sample, comprising:
contacting a sample from a subject with the isolated molecule of claim 1, and
detecting binding of the isolated molecule to components in the sample.

44. The method of claim 43 wherein the isolated molecule is bound to a detectable label.

45. A method of detecting cancer cells in a human, comprising:
contacting a subject with the isolated molecule of claim 1, and
detecting binding of the isolated molecule to particular organs of the subject.

46. The method of claim 45 wherein the isolated molecule is bound to a detectable label.

47. A method of selecting from a nucleic acid library an scFv or Fab or T cell receptor that specifically binds to a first form of a peptide portion of a protein or full length protein, wherein the first form comprises a mutant residue, wherein the scFv or Fab or TCR does not specifically bind to the peptide or full length protein in its wild-type form, the method comprising a step of:
positively selecting for scFv or Fab or T cell receptors that bind to the first form in the presence of a competitor second form of the peptide portion or full length protein, wherein the second form is selected from the group consisting of a wild-type form and a peptide or full length protein with a different mutant residue than the first form.

48. The method of claim 47 which comprises the steps of:
a. negatively selecting for scFv or Fab or T cell receptors that bind to unfolded first form;
b. positively selecting for scFv or Fab or T cell receptors that bind to folded first form;
c. positively selecting for scFv or Fab or T cell receptors that bind to the first form in the presence of the second form;
d. negatively selecting for scFv or Fab or T cell receptors that bind to the second form;
e. positively selecting for scFv or Fab or T cell receptors that bind to the first form.

49. The method of claim 48 wherein pairs of steps (a) and (b), (a) and (c), and (d) and (e) are performed a plurality of times.

50. The method of claim 49 wherein after each pair of positive and negative selection steps, remaining scFv or Fab or T cell receptors are amplified.

51. The method of claim 49 wherein during successive performance of step (c), amounts of said first form and said second form are varied so that ratio of second form to said first form increases.
